(19) United States
(12) Patent Application Publication
(10) Pub. No.: US 2026/0315243 A1
(43) Pub. Date: Oct. 15, 2026

(54) FIXATION SYSTEM AND METHOD FOR FIXING PROSTHETIC IMPLANT COMPONENTS FOR KNEE AND HIP ARTHROPLASTIES

(71) Applicant: Hugo Domínguez Fernández, Higüey (DO)

(72) Inventor: Hugo Domínguez Fernández, Higüey (DO)

(21) Appl. No.: 19/174,518

(22) Filed: Apr. 9, 2025

(51) Int. Cl. A61F 2/46; A61B 17/16; A61B 17/17; A61B 90/00; A61F 2/30; A61F 2/34; A61F 2/36

(52) U.S. Cl. CPC A61F 2/4609; A61B 17/1666; A61B 17/1746; A61F 2/30734; A61F 2/34; A61F 2/3662; A61B 2090/363; A61B 2090/364; A61F 2002/30736; A61F 2002/3432; A61F 2002/365; A61F 2002/4631

(57) ABSTRACT

Uncemented bone fixation systems and techniques can be used for fixing prosthetic implant components to bone. In some examples, the systems and techniques utilize osteomorphically arranged interference screws. The systems and techniques can be used in various orthopedic applications, including knee and hip arthroplasties.

### OBJECT OF THE INVENTION

The present invention relates to an uncemented bone fixation system and method for fixing prosthetic implant components by means of using osteomorphically arranged interference screws. More particularly, the invention relates to the field of knee and hip arthroplasties.

### BACKGROUND OF THE INVENTION

Arthroplasty is a surgical procedure whereby a surgeon removes injured cartilage and bone from the joint of a patient and replaces them with artificial prosthetic implants.

The main arthroplasties, by number of annual surgeries, are knee arthroplasties and hip arthroplasties. It is estimated that, by 2030, the number of total knee replacement (TKR) surgeries in the US will reach 1.28 million annual surgeries, and the number of total hip replacement (THR) surgeries in the US will reach 635,000 annual surgeries (American Academy of Orthopaedics Surgeons).

Prosthetic implant components used in arthroplasties may be cemented or uncemented.

Uncemented implants have the advantage of providing an osseointegration fixation of the prosthetic implant component that is more natural than that provided by cement. Since uncemented implants use the natural bone of the patient to keep them in place, it is believed that they will last longer and form a more permanent attachment with the bones of the patient than cemented implants.

Moreover, in uncemented implants, the duration of surgery lasts about thirty minutes less than surgery with cemented implants, which entails a significant advantage for the patients who have to remain under anesthesia in the operating room for a shorter time.

With respect to cemented implants, the main drawback is that cement is toxic and entails the risk of causing intraoperative cardiopulmonary problems and pulmonary embolism that are sometimes fatal for patients. The risk of thrombosis following total knee arthroplasty with cemented implants is between 40% and 80%. A 2017 study (Journal of Bone and Joint Surgery) revealed that the main cause of death 90 days after knee arthroplasty operations was cardiac ischemia.

Moreover, although implants cemented a priori are cheaper, if the longer duration of the surgery and the price derived from the material required during the surgery (obturators, cement, and cementation sets) are taken into account, the total expense may be higher than that for uncemented implants.

Another drawback of cemented implants is that the polymerization reaction of cement is highly exothermic and its extravasation can cause lesions in neighboring vascular and nerve structures of the patient.

An additional drawback of cemented implants is that cement ages and fractures. Therefore, the percentages of aseptic mobilization of cemented implant components are more common as the follow-up time increases. In contrast, uncemented implant components promote a “biological fixation” since they favor the osseointegration of the prosthetic implant component in the bone which requires a longer consolidation process, but does not deteriorate over time.

Another drawback to be taken into account is that the revision of mobilized cemented implants tends to be complicated since bone losses are greater and increase when there is a need to extract the cement.

Furthermore, recent research highlights that cemented knee replacements in younger patients may not last as long as replacements in older patients, simply due to their increased activity and demand they place on the implants and cement, which can lead to premature loosening.

Taking into account these risks and complications associated with cemented implants, in 2021, 95.2% of hip arthroplasties (THA) were carried out in the United States with uncemented implants (2022 AAOS American Joint Replacement Registry).

However, the percentage of uncemented knee implants only reached 18.8% (2022 AAOS American Joint Replacement Registry). This is because current uncemented knee implant systems have higher revision rates than cemented knee implants, mainly due to instability and/or aseptic loosening of the implant.

Moreover, although long bone anchors (those inserted into the medullary canal) improve the mechanical stability of the prosthetic implant component, it is at the expense of a reduction in the periprosthetic bone density of the patient, a risk of subsidence and loosening of the bone anchor, a risk of periprosthetic fracture and pain in the extremity. For example, recent studies reveal that between 16-33% of patients claim to suffer chronic pain after knee arthroplasty surgery (EFORT Open Reviews, “Chronic Pain After Total Knee Arthroplasty,” Aug. 16, 2018).

For these reasons, the current trend in tibial knee implant components and femoral hip implant components is to shorten the bone anchor. Short bone anchors do not invade the bone canal, thereby eliminating pain and successfully distributing stresses in a more physiological manner, with decreased diaphyseal changes which cause bone loss due to distortion in the original load distribution and which can lead to periprosthetic fractures in the long term.

Furthermore, it should be taken into consideration that the implantation of short anchors is easier and they preserve the tissue (hard and soft) to a greater extent, reduce surgery time, and reduce blood loss. As a result, the risk of thromboembolism decreases since short anchors allow total support and early ambulation in the immediate postoperative period, i.e., they allow the patient to adopt an upright position (to stand) earlier and to start walking with the help of crutches. Furthermore, as a result of the patients being able to start rehabilitation within a short period of time, muscle mass loss is minimized.

However, implant components comprising uncemented short anchors have a smaller osseointegration surface than long bone anchor implant components. This smaller surface receive the same stresses, so tension points that cause certain micromobility which prevents suitable bone tissue growth around the implant emerge, with fibrous tissue being formed instead which will eventually cause the macroscopic mobilization or micromobilization (stable fibrous fixation) of the implant.

Once a prothesis has become loose, there is no way to fix it again, with revision surgery being the only viable alternative to cure pain and functional incapacity that occurs.

The aseptic loosening of prosthetic implants is mainly caused by the failure to obtain a suitable primary stability when placing said implants during surgery, with this being understood as the strength and rigidity of the implant-bone attachment before osseointegration of the implant.

Without immediate primary stability, it is impossible for patients to adopt an upright position and to start walking earlier, which are very important for a successful rehabilitation protocol.

To solve this problem, there are designs in the state of the art which, for the purpose of improving the primary stability of uncemented implant components, incorporate axial fixation screws.

Once screwed in, axial fixation screws comprise two different longitudinal portions: a first longitudinal portion screwed into the implant component and a second longitudinal portion screwed into the bone.

For example, document U.S. Pat. No. 10,864,082 describes a tibial knee implant which comprises axial fixation screws.

More particularly, the tibial implant component comprises a short bone anchor around a central axis and axial fixation screws comprising threading axes parallel to said central axis.

This parallel configuration is logical from a solely mechanistic perspective which, for the purpose of maximizing the axial fixation force provided by the screws, places the threading axes of the screws, perpendicular to the plane of the tibial tray and parallel to one another.

The problem with the fixation system by means of axial fixation screws is that, on one hand, the bone anchor supports more load as it becomes osseointegrated, so the load supported by the screws decreases. On the other hand, the parallel arrangement alters the original force lines.

According to Wolff's law, this causes bone mineral density loss (stress shielding) both in the trabecular bone, around the screws, and in the cortical bone and dense trabecular bone.

Bone mineral density loss around the screws generates micromovements which, over time, can lead to aseptic loosening of the previously osseointegrated implant component. Furthermore, bone mineral density loss in the bone cortex and dense trabecular bone can cause periprosthetic fractures.

Both of the problems can cause high rates of aseptic loosening of uncemented knee implants, so, despite the serious complications associated with the use of cement set forth above, about 80% of knee arthroplasties are still carried out at present by means of cemented implant components.

In a second example, document US 2018 0344466 describes an acetabular component of a hip implant which also comprises axial fixation screws.

In this second document, also with a solely mechanistic perspective, the threading axes of the screws are arranged radially with respect to the prosthetic body, which is hemispherical.

This configuration of the screws may cause osteolysis around same. Furthermore, the radial arrangement alters the original force lines, which entails the risk of suffering variations in the bone density of the patient, which can lead to loosening of the acetabular component in the medium or long term. Lastly, due to their radial arrangement, some of the screws follow transcortical trajectories, which entails the risk of causing neurovascular damage to the patient.

However, despite these associated problems, more than 95% of hip arthroplasties are carried out at present by means of uncemented implant components, and in fact, the main manufacturers today sell acetabular fixation systems with axial fixation screws.

However, according to the 2021 annual report of the National Joint Registry of the United Kingdom, between 10% and 20% of hip implants require revision within 1 to 10 years.

It should also be taken into account that the volume of arthroplasties is increasingly higher with increasing life expectancy in a globally aging population, and this leads to increasing revision rates. For men up to 50 years of age, after total hip or knee replacement, the risk of a revision surgery increases to 35% (Lee E Bayliss et al. Lancet 2017: “Risk of implant revision after total replacement of the hip or knee”).

The objective of the present invention is to reduce the current revision rates of uncemented implants and to prolong the time which said implants will last by providing to that end an uncemented bone fixation system and method for fixing prosthetic implant components for lower extremity arthroplasties, solving the problems described above.

### DESCRIPTION OF THE INVENTION

The present invention proposes a solution to the preceding problems by means of a bone fixation system for fixing a prosthetic implant component to a bone of a patient in knee or hip arthroplasties according to claim 1, a bone fixation assembly for fixing prosthetic implant components to the tibia and to the femur of a patient in knee arthroplasties according to claim 15, a bone fixation assembly for fixing prosthetic implant components to the femur and to the pelvis of a patient in hip arthroplasties according to claim 16, and a bone fixation method for fixing a prosthetic implant component to a bone of a patient in knee or hip arthroplasties in claims 17 and 18. The dependent claims define preferred embodiments of the invention.

A first inventive aspect provides a bone fixation system for fixing a prosthetic implant component to a bone of a patient in knee or hip arthroplasties, wherein said system comprises:
a prosthetic implant component comprising:
   a prosthetic body comprising a first face, configured to be coupled at least partially to a prosthetic insert, and a second face, configured to at least partially contact with the bone,
   a bone anchor, configured to be implanted in the bone, comprising a proximal end, attached to the second face of the prosthetic body, and a distal end, and
   a plurality of through conduits, each of which is oriented according to a longitudinal axis, and each of which comprising:
      a first portion, formed as a closed conduit in the prosthetic body going through said prosthetic body from the first face to the second face and, optionally, partially going through the bone anchor, and
      a second portion, after the first portion, which is formed as an open conduit in the bone anchor defining a fixing area,
   wherein:
   the first portion and/or the second portion comprise a first threading region, and
   the longitudinal axes of at least two of the plurality of through conduits form an angle greater than zero degrees and less than ninety degrees, the angle being closed in the proximal-distal direction,
a plurality of interference screws, each of which
   comprises a second threading region, complementary to the first threading region of a corresponding through conduit, and
   is configured to be screwed into a corresponding through conduit, and wherein, when the bone anchor is implanted in a bone housing carved in the bone, the prosthetic implant component is fixed to the bone when the interference screws are screwed into the through conduits going through the fixing areas.

The bone fixation system of the first inventive aspect is intended for fixing a prosthetic implant component, with it being understood as a piece used to replace a joint in a patient's body with a bone from said patient. In particular, the bone fixation system can be used in knee or hip arthroplasties.

The system comprises a prosthetic implant component which in turn comprises a prosthetic body, a bone anchor, and a plurality of through conduits.

The prosthetic body comprises a first face configured, sized, and shaped to be coupled at least partially to a prosthetic insert. Throughout this document, prosthetic insert shall be understood to mean a piece of a prosthetic joint assembly to be placed between the two components of said prosthetic joint assembly to reduce the friction of the assembly.

The prosthetic body also comprises a second face configured, sized, and shaped to at least partially contact the bone of the patient. In one embodiment, the second face is configured, sized, and shaped not only to contact the bone of the patient, but also to be coupled with said bone.

The bone anchor, the piece of the prosthetic implant component configured to be implanted in the bone of the patient, is attached to this second face. Throughout this document, bone anchor shall be understood to mean any longitudinal portion of the prosthetic implant component suitable to be inserted and housed in a bone housing inside the bone, which has been previously carved to that end in said bone.

This bone anchor comprises a proximal end and a distal end. Throughout this document, the distal end of the at least one bone anchor shall be understood to mean the end of the bone anchor suitable to be inserted into the bone first. Moreover, the proximal end of the at least one bone anchor shall be understood to mean the end of the bone anchor suitable to be inserted into the bone last.

The bone anchor comprises a geometry with a proximal cross-section having an area greater than the area of the distal cross-section; for example a keel-shaped geometry, and/or a frustoconical geometry, and/or an oval frustoconical geometry, and/or a pyramidal geometry, and/or wedge-shaped geometry, and/or an arrow tip-shaped geometry.

The prosthetic implant component further comprises a plurality of through conduits each oriented in accordance with a longitudinal axis. Each through conduit comprises two portions:
A first portion formed as a 360° closed conduit which goes through the prosthetic body from the first face to the second face. Optionally, this first portion (closed conduit) can be partially prolonged through the bone anchor, and
a second portion formed as an open conduit extending longitudinally through the bone anchor. The second portion is after the first portion and defines a fixing area.

The first portion, the second portion, or both portions comprise a first threading region. Throughout this document, threading region shall be understood to mean a series of thread segments belonging to one or more thread helices.

In an embodiment in which both portions comprise the first threading region, the threading region of the first portion is a complete thread and the threading region of the second portion is a truncated thread, with the complete and truncated threads being coaxial and with both threads having the same lead as the lead of the corresponding interference screw.

Throughout this document, the lead of a thread shall be understood to mean the distance between two consecutive profiles corresponding to the same helix, measured in the direction of the threading axis.

The longitudinal axes defining the orientation of at least two of the through conduits form an angle greater than zero degrees and smaller than ninety degrees. Said angle is closed in the proximal-distal direction, that is, from the prosthetic body towards the distal end of the bone anchor.

Moreover, the system comprises a plurality of interference screws, one for each through conduit existing in the prosthetic component.

In the state of the art, interference screws are screws used mainly in repairing cruciate ligaments. These interference screws are screwed between the soft tissue graft used for the repair and the wall of the bone tunnel surrounding same, performing fixation by interference.

Throughout this document, interference screw shall be understood to mean a screw which is suitable to be screwed into its through conduits when the prosthetic implant component is implanted in the bone, being intercalated between the bone anchor and the bone to provide fixation by interference which secures the bone anchor in a specific position with respect to the bone.

Each interference screw comprises a second threading region which is complementary to the first threading region of its corresponding through conduit.

Throughout this document, it shall be understood that if the first threading region has a female thread (in the first portion of a through conduit) and/or a truncated female thread (in the second portion of a through conduit), the complementary thread of the second threading region (in the interference screw) is a male thread, or vice versa.

In this way, the interference screws can be screwed into their corresponding through conduit. Therefore, when the bone anchor is implanted or housed in a bone housing carved in the bone, the interference screws allow fixing the prosthetic implant component to the bone, being screwed in the through conduits going through the fixing areas thereof.

In the fixation system of the invention, fixation by interference provided by the interference screws is particularly effective since they are osteomorphically arranged and work synergistically.

Throughout this document, osteomorphic arrangement of the interference screws shall be understood to mean the arrangement that said interference screws acquire by virtue of the angle that is greater than zero degrees defined between at least two of the plurality of through conduits suitable for housing them.

Screws arranged parallel to one another of the state of the art provide a significant axial force and high mechanical fixation, but at the expense of an alteration of the original force lines of the bone.

In contrast, osteomorphic arrangement of the interference screws of the invention allows the fixing areas to be consistent with the main force lines of the bone before arthroplasty, which are not parallel to one another, but extending mainly along the cortical bone and/or along the dense subcortical trabecular bone, according to the particular morphology of each bone.

Bone morphology determines the force lines and vice versa. In the tibia, in the femur, and in the ilium, the main force lines are not parallel to one another, but are stress lines, or trabecular stress groups, aligned with the cortical bone and with the dense trabecular bone, which, under normal conditions, support most of the mechanical load of the bone.

If the force lines are altered, bone morphology is altered, with this alteration being one of the most common causes of periprosthetic fractures and osteolysis around the screws.

Osteomorphic arrangement of the interference screws solves these two complications associated with the use of parallel axial fixation screws.

Moreover, as discussed above, interference screws work synergistically. Osteomorphic arrangement of the interference screws of the invention reduces the axial force that said interference screws would provide if they were arranged in parallel. However, each interference screw, in addition to fixing the prosthetic implant component to the bone by means of axial force, provides an expansive force that reinforces the fixation of the prosthetic implant component in the area opposite the fixing area. In this way, when all the screws work synergistically, the sum of axial and expansive forces is equal to or greater than the axial fixation force provided by the fixation screws of the state of the art.

As a result thereof, the fixation system of the invention is an uncemented system which does not require a diaphyseal anchor, providing the immediate primary stability required for the patients to adopt an upright position and to start walking earlier, with both issues, as mentioned above, being very important for a successful rehabilitation protocol.

Another advantage derived from the fixation system of the invention is that it is particularly suitable for lower extremity arthroplasties in young and active patients since, by decreasing the risk of osteolysis around the interference screws and decreasing the risk of periprosthetic fractures, it decreases the risk of a revision surgery after total hip or knee replacement.

In one embodiment, the second portion of each through conduit of the plurality of through conduits is formed as an open conduit the cross-section of which is in the shape of a circumference arch with an angle Φ equal to or greater than 180°.

Throughout the document, a circumference arch shall be understood to mean the arch of a circle between a pair of different points. In this way, the open conduit of this embodiment has a cross-section in a semicircle shape (when the angle Φ is 180°) or a partial circle shape (when the angle Φ is greater than 180°), with the open conduit having a concave inner surface corresponding with the fixing area.

In one embodiment, the angle Φ of the circumference arch of the cross-section of the open conduit decreases in the proximal-distal direction; i.e., the angle Φ is greater at the proximal end of the second portion of the through conduit (the one closest to the prosthetic body) than at its distal end (the one farthest away from the prosthetic body).

For example, an open conduit with a proximal cross-section in the shape of a circumference arch with an angle Φ equal to 359° and with a distal cross-section in the shape of a circumference arch with an angle Φ equal to 180°.

In one embodiment, the angle Φ of the circumference arch of the cross-section of the open conduit gradually decreases in the proximal-distal direction, describing a curve.

In one embodiment, the open conduit comprises two sub-portions. In the first sub-portion, the angle Φ of the circumference arch which describes the cross-section thereof gradually decreases from the proximal end of the conduit (which coincides with the proximal end of the first sub-portion) to the distal end of said first sub-portion. In the second sub-portion, after the first sub-portion, the angle Φ of the circumference arch which describes the cross-section of the open conduit remains constant between the two ends thereof.

For example, an open conduit with:
a first sub-portion, wherein the angle Φ is equal to 359° at its proximal end and 180° at its distal end; and
a second sub-portion, along which the angle Φ remains constant, with Φ being equal to 180°.

In all these embodiments, the fixing area is the concave inner surface of the open conduit.

In all these embodiments, the advantage of having an open conduit in which the angle Φ is equal to or greater than 180° is that the occurrence of dead points around the interference screws is prevented. Furthermore, the convergence of the fixing areas in the proximal-distal direction also prevents the existence of dead points around the bone anchor. Throughout this document, dead points around the interference screws and/or around the bone anchor shall be understood to mean those areas of the surface of the screw and/or of the bone anchor that do not receive load when the patient is standing or walking, and in which osteolysis, i.e., bone density loss, may thus occur. As a result of osteolysis, loosening of the implant takes place.

In a particular embodiment, the angle formed by the longitudinal axes of at least two through conduits of the plurality of through conduits is in the range of 5-65 degrees.

Advantageously, restoration of the main force lines of the original joint is particularly effective when the angle between through conduits is in the range of 5-65 degrees. As mentioned above, these force lines are osteomorphic force lines in the sense that they are not parallel to one another, but fundamentally arranged according to the particular morphology of each bone.

Moreover, in the range of 5-65 degrees, the expansive force generated by the interference screws of the invention is sufficient to compensate for axial force loss that occurs in comparison with the axial force generated by the parallel interference screws of the state of the art.

In one embodiment, at least one portion of the first inner face of the prosthetic body comprises grooves, and/or channels, and/or depressions, and/or any other type of notching and/or ribbing that allow bone filling. In this way, along with the action of the at least two interference screws, resistance to posteromedial and posterolateral translation forces, and/or anteroposterior translation forces, and/or flexion-extension forces, and/or abduction-adduction forces, and/or internal-external rotation forces, and/or compression forces that act on the prosthetic implant component increases.

In a particular embodiment,
the first portion of each through conduit comprises a proximal part and a distal part, wherein
   the distal part comprises the first threading region, and
   the proximal part has a frustoconical section; and
each interference screw comprises a frustoconical head complementary to the proximal part of the first portion.

In this embodiment, the proximal part of the first portion of a through conduit is that which is the closest to the first face of the prosthetic body, whereas the distal part is that which is the closest to the second face of the prosthetic body.

As a result of the particular shape of the frustoconical heads of the through conduit and of the interference screw, as well as the arrangement of the threading regions:
the interference screws are screwed into the through conduits in a simple and effective manner, and
a tight fitting which reduces the micromobility of the interference screws, and therefore the risk of the implant component loosening due to this, is provided.

In a particular embodiment, the thread of the plurality of interference screws is blunt, squared, rounded, or trapezoidal.

Advantageously, the threads of this embodiment are suitable to be screwed into the through conduits without damaging the dense trabecular bone extending between each fixing area and the cortical bone. More particularly, blunt thread protects inner bone cortex, allowing the interference screws to be screwed into the most dense portion of the trabecular bone, close to the cortex, while providing at the same time an improved stress distribution.

In a particular embodiment, the system further comprises:
a screwdriver element configured to screw the interference screws to the prosthetic implant component, and/or
a bone preparation element configured to carve the bone and create a bone housing sized and configured to house the bone anchor.

In this embodiment, the system further comprises a screwdriver element configured, sized, and shaped to enable screwing the interference screws to the prosthetic component, by screwing them into the through conduits.

Additionally or alternatively, the system comprises a bone preparation element with which a surgeon can carve the bone in which the prosthetic implant component will be fixed, creating to that end a bone housing with suitable dimensions to enable housing the bone anchor therein.

Examples of bone preparation elements are: cutting guides, drilling guides, reaming guides, oscillating saws, circular saws, drills, compacting drills, bits, osteotomes, chisels, scrapers, reamers, rongeurs, impactors, countersinks, dilators, taps, thread forming devices, or temporary prosthetic components.

In a particular embodiment, the plurality of interference screws comprise a metric of between 6 mm and 20 mm, and a length of between 15 mm and 100 mm.

Throughout the document, the metric of an interference screw shall be understood to mean its diameter.

In one embodiment, the plurality of interference screws comprise a cylindrical geometry with a slight apical conicity and/or a rounded tip.

In one embodiment, the second threading region of the plurality of interference screws has a progressive thread profile, which facilitates the insertion of each interference screw into its corresponding through conduit, acting as a bone compactor which increases primary stability.

In one embodiment, the plurality of interference screws are cannulated, comprising a hollow central shaft for improved torque transmission.

In one embodiment, the plurality of interference screws comprise simple spirals. In another embodiment, to improve grip on the bone, the plurality of interference screws comprise double or triple spirals.

In one embodiment, the plurality of interference screws are headless interference screws. In another embodiment, the plurality of interference screws each comprises a head. In a more particular embodiment, the plurality of interference screws comprise a beveled head, such that once the interference screw is screwed into the corresponding through conduit, the head of the screw is flush with the first face of the prosthetic body.

In one embodiment, to improve grip on the bone, the plurality of interference screws comprise macroscopic grooves between the threads and/or in the crests of the threads, so as to increase the contact surface with the bone and thereby favor their osseointegration.

In one embodiment, the plurality of interference screws comprise a slightly conical and/or rounded tip.

In one embodiment, for an improved distribution of the tightening torque, the plurality of interference screws comprise a cruciform or stardrive cavity.

In one embodiment, the screwdriver element and the plurality of interference screws are designed to be automatically secured to one another.

In one embodiment, the maximum torque recommended for tightening the plurality of interference screws is between 20 Ncm and 100 Ncm.

In one embodiment, the prosthetic implant component and/or the plurality of interference screws are manufactured and/or are coated with at least one of the following biomaterials: titanium alloys, porous titanium alloys; COCrMo alloys; CoCrMoCaP alloys; tantalum trabecular metals; porous or non-porous ceramic materials, such as alumina (Al₂O₃) and zirconium oxide (ZrO₂); Peek (polyether-ether-ketone), Pekk (polyether-ketone-ketone), bone allograft, hydroxyapatite (HA) (C₁₀(PO₄)₆ OH₂).

Advantageously, these materials improve at least one of: biocompatibility, bioactivity, elasticity, load distribution, osseointegration, strength, and durability.

In one embodiment, the prosthetic implant component comprises a porous structure manufactured by means of one of the following techniques: additive manufacturing (AM), selective laser manufacturing (SLM), or electron beam melting (EBM).

In one embodiment, the prosthetic implant component and/or the plurality of interference screws are manufactured in Peek subjected to a treatment to increase its bioactivity and/or in a bioactive Peek compounds.

Throughout the document, Peek shall be understood to mean a non-cytotoxic, inert material which minimizes the risk of local inflammatory reactions, with a low density and low thermal and electrical conduction in comparison with existing implant materials, and with an elasticity modulus E-modulus of 4 GPa, comparable to the cortical bone. Peek is furthermore radiotransparent and compatible with common imaging technologies for improved visualization during implantation and during post-surgery follow-up of the implant osseointegration process.

In one embodiment, the prosthetic implant component is manufactured in a bioactive metallic biomaterial, for example titanium, and the plurality of interference screws are manufactured in Peek subjected to a treatment to increase its bioactivity and/or in a bioactive Peek compound.

In one embodiment, the prosthetic implant component is manufactured in Peek subjected to a treatment to increase its bioactivity and/or in a bioactive Peek compound, and the plurality of interference screws are manufactured in a bioactive metallic biomaterial, for example titanium.

In one embodiment, the prosthetic implant component and/or the plurality of interference screws further comprise piezoelectric nanoparticles and/or devices for the collection and intelligent transmission of data and/or for the administration of medicinal products to improve the mechanical and/or biological performances of current implants.

In a particular embodiment, the prosthetic implant component is a tibial prosthetic implant component and:
the first face of the prosthetic body is configured to be coupled at least partially to a tibiofemoral insert and the second face is configured to contact a tibial bone resection plane,
the bone anchor comprises an anterocentral tibial piece, a posteromedial tibial piece, and a posterolateral tibial piece, and
the plurality of through conduits comprises an anterocentral through conduit, a posteromedial through conduit, and a posterolateral through conduit, and
   wherein
the longitudinal axes of the posterolateral through conduit and the posteromedial through conduit form an angle between 30 and 65 degrees,
the longitudinal axis of the anterocentral through conduit forms, with each of the longitudinal axes of the posterolateral through conduit and the posteromedial through conduit, an angle between 15 and 45 degrees, and
   wherein the bone fixation system comprises three interference screws.

This embodiment describes a bone fixation system for knee arthroplasties with three interference screws, wherein the prosthetic implant component is a tibial component.

In this embodiment, the first face of the prosthetic body comprises a tibial tray configured, sized, and shaped to be coupled with a tibiofemoral insert; and the second face is configured, sized, and shaped to contact or to be coupled to a tibial bone resection plane.

Furthermore, the bone anchor comprises three pieces, preferably attached to one another: an anterocentral tibial piece, a posteromedial tibial piece, and a posterolateral tibial piece. A different through conduit is formed partially on each of said pieces. Therefore, the prosthetic implant component comprises three through conduits: an anterocentral through conduit, a posteromedial through conduit, and a posterolateral through conduit.

In one embodiment, the pieces of the bone anchor are attached to one another. In another embodiment, the pieces of the bone anchor are not attached to one another, such that each is independent of the others.

Throughout this document, the anterocentral, posterolateral, and posteromedial through conduits of a prosthetic implant component for arthroplasties shall be understood to mean the through conduits which would be positioned anterocentral, posterolateral, and posteromedial, respectively, in the anatomical direction, once the prosthetic implant component is implanted in the bone of a patient in an upright position.

In this embodiment, the longitudinal axes of the posterolateral through conduit and the posteromedial through conduit form an angle between 30 and 65 degrees, and the longitudinal axis of the anterocentral through conduit forms, with each of the longitudinal axes of the posterolateral through conduit and the posteromedial through conduit, an angle between 15 and 45 degrees.

Advantageously, this embodiment, by virtue of the osteomorphic arrangement of the interference screws, solves two important problems associated with current uncemented fixation systems for tibial knee implant components:
it prevents the development of osteolysis around the interference screws as the tibial knee implant component becomes osseointegrated, and
it promotes the physiological transfer of axial and torsional load between the femur and the tibia, similar to the conditions prior to surgery, which prevents bone density loss in the tibial epiphysis, and therefore decreases the risk of post-surgery periprosthetic fractures.

The convergence of the fixing areas in the proximal-distal direction is possible by means of the proximal-distal convergence of bone anchors, which ensures that there are no dead points (that do not receive loads) in the bone anchor or in the interference screws when the patient is standing or walking. Therefore, based on Wolff's law, the bone around the bone anchor and around the interference screws is prevented from losing density as the bone anchor and the interference screws become osseointegrated.

In a particular embodiment, each of the pieces of the bone anchor is formed as a pyramid frustum or a cone frustum.

In a particular embodiment, the prosthetic implant component is a knee femoral prosthetic implant component and:
the first face of the prosthetic body is configured to be coupled at least partially to a tibiofemoral insert and the second face is configured to contact a femoral condyle bone resection plane,
the plurality of through conduits comprises a medial through conduit and a lateral through conduit, and
   wherein the longitudinal axes of the medial through conduit and of the lateral through conduit form an angle between 15 and 45 degrees,
   and wherein the bone fixation system comprises two interference screws.

This embodiment describes a bone fixation system for the femoral prosthetic implant component for knee arthroplasties, with two interference screws.

In this embodiment, the first face of the prosthetic body is a condylar face configured, sized, and shaped to be coupled in a rotary-sliding manner with a tibiofemoral insert; and the second face is configured, sized, and shaped to be coupled to or to contact a femoral condyle bone resection plane.

Moreover, the femoral knee implant component comprises two through conduits: a medial through conduit and a lateral through conduit.

Throughout this document, the medial and lateral through conduits of a prosthetic implant component for arthroplasties shall be understood to mean the through conduits which would be positioned medial and lateral, respectively, in the anatomical direction once the prosthetic implant component is implanted in the bone of a patient in an upright position.

In this embodiment, the longitudinal axes of the through conduits form an angle between 15 and 45 degrees.

Advantageously, this embodiment, by virtue of the osteomorphic arrangement of the interference screws, solves two important problems associated with current uncemented fixation systems for femoral knee implant components:
it prevents the development of osteolysis around the interference screws as the femoral knee implant component becomes osseointegrated, and
it promotes the physiological transfer of axial and torsional load between the femur and the tibia, similar to the conditions prior to surgery, which prevents bone density loss in the femoral epiphysis, and therefore decreases the risk of the implant component loosening and/or of post-surgery periprosthetic fractures.

In a particular embodiment, the bone anchor comprises a piece formed as a pyramid frustum which, with the exception of its base, comprises four concave faces.

In this embodiment, the bone anchor comprises four concave faces: a medial face, a lateral face, an anterior face, and a posterior face. In particular, the second portion of the medial through conduit extends along the medial face of the bone anchor and the second portion of the lateral through conduit extends along the lateral face of the bone anchor.

Throughout this document, the medial, lateral, anterior, and posterior faces of a bone anchor of a prosthetic implant component for arthroplasties shall be understood to mean the faces of the bone anchor which would be positioned medial, lateral, anterior, and posterior, respectively, in the anatomical direction once the prosthetic implant component is implanted in the bone of a patient in an upright position.

Advantageously, the concave faces of the bone anchor allow preserving a large amount of intercondylar bone stock, while providing at the same time a large osseointegration surface, all of which reinforces the primary and secondary fixations of the prosthetic implant component.

Furthermore, once the implant component is implanted and fixed, the convergence of the fixing areas ensures that there are no dead points in the bone anchor (likewise convergent) or in the interference screws. Therefore, based on Wolff's law, the bone around the bone anchor and around the interference screws is prevented from losing bone density as the bone anchor and the interference screws become osseointegrated.

In a particular embodiment, the prosthetic implant component is a hip femoral prosthetic implant component, and:
the second face is configured to contact a femoral bone resection plane,
the first face of the prosthetic body is configured to be coupled at least partially to a femoroacetabular insert,
the plurality of through conduits comprises a lateral through conduit and a posterior through conduit, and
   wherein the longitudinal axes of the lateral through conduit and of the posterior through conduit form an angle between 5 and 45 degrees,
   and wherein the bone fixation system comprises two interference screws.

This embodiment describes a bone fixation system for the femoral prosthetic implant component for hip arthroplasties, with two interference screws.

In this embodiment, the first face of the prosthetic body is configured, sized, and shaped to be coupled in an articulated manner to a femoroacetabular insert; and the second face is configured, sized, and shaped to be coupled to or to contact a femoral bone resection plane.

Moreover, the femoral component for hip arthroplasty comprises two through conduits: a lateral through conduit and a posterior through conduit.

Throughout this document, the lateral and posterior through conduits of a prosthetic implant component for arthroplasties shall be understood to mean the through conduits which would be positioned lateral and posterior, respectively, in the anatomical direction once the prosthetic implant component is implanted in the bone of a patient in an upright position.

In this embodiment, the longitudinal axes of the through conduits form an angle between 5 and 45 degrees.

Advantageously, this embodiment, by virtue of the osteomorphic arrangement of the interference screws, solves two important problems associated with current uncemented fixation systems for femoral hip implant components:
it prevents the development of osteolysis around the fixation screws as the femoral hip implant component becomes osseointegrated, and
it promotes the physiological transfer of axial and torsional load between the femur and the ilium, similar to the conditions prior to surgery, which prevents bone density loss in the head of the femur, and therefore decreases the risk of the implant component loosening and/or of post-surgery periprosthetic fractures.

Another advantage of the fixation system of this embodiment is that it allows the fixing area of the lateral through conduit to be positioned in the lateral cancellous bone of the greater trochanter, such that, since the greater trochanter comprises cancellous bone in greater number and better quality, improved primary and secondary fixations of the hip femoral prosthetic implant component along the main tensile group are achieved. Additionally, the fixing area of the posterior through conduit increases the resistance of the fixation to subsidence, to torsional forces, and to horizontal movements, which advantageously helps to eliminate micromovements at the level of implant-bone interface.

In a particular embodiment, the prosthetic implant component is an acetabular prosthetic implant component, and:
the prosthetic body is an acetabular prosthetic body formed as an acetabular cup comprising a circumference flange comprised in plane π,
the second face of the acetabular prosthetic body is configured to contact an acetabular bone,
the first face of the acetabular prosthetic body is configured to be coupled at least partially to a femoroacetabular insert,
the plurality of through conduits comprises an anterior through conduit, a medial-superior through conduit, a medial-inferior through conduit, and a posterior through conduit, and
   wherein
the longitudinal axes of the medial-inferior through conduit and the medial-superior through conduit form an angle between 15 and 60 degrees,
the longitudinal axis of the medial-superior through conduit forms an angle between 30 and 90 degrees with respect to plane π, and
   wherein the bone fixation system comprises four interference screws.

This embodiment describes a bone fixation system for hip arthroplasties with four interference screws, wherein the prosthetic implant component is an acetabular component.

In this embodiment, the prosthetic body is an acetabular cup, with a first concave face configured, sized, and shaped to be coupled with a femoroacetabular insert; and the second face is configured, sized, and shaped to be coupled to or to contact an acetabular bone. Furthermore, the prosthetic body comprises a circumference flange comprised in plane π.

The acetabular component comprises four different through conduits: an anterior through conduit, a medial-superior through conduit, a medial-inferior through conduit, and a posterior through conduit.

Throughout this document, the anterior, medial-superior, medial-inferior, and posterior through conduits of a prosthetic implant component for arthroplasties shall be understood to mean the through conduits which would be positioned anterior, medial-superior, medial-inferior, and posterior, respectively, in the anatomical direction once the prosthetic implant component is implanted in the bone of a patient in an upright position.

In this embodiment, the longitudinal axes of the medial-inferior and medial-superior through conduits form an angle between 15 and 60 degrees, and the longitudinal axis of the medial-superior through conduit forms an angle between 30 and 90 degrees with respect to plane π.

Advantageously, this embodiment, by virtue of the osteomorphic arrangement of the interference screws, solves two important problems associated with current uncemented fixation systems for acetabular implant components:
it prevents the development of osteolysis around the fixation screws as the acetabular implant component becomes osseointegrated, and
it promotes the physiological transfer of axial and torsional load between the femur and the ilium, similar to the conditions prior to surgery, which prevents bone density loss in the ilium, and therefore decreases the risk of the implant component loosening and/or of post-surgery periprosthetic fractures.

In a particular embodiment, the bone anchor comprises a piece formed as a pyramid frustum inclined with respect to plane π, which comprises eight concave faces in its medial cross-section.

Advantageously, in this embodiment, the concave faces of the bone anchor allow preserving a large amount of iliac bone stock, while providing at the same time a large osseointegration surface, all of which reinforces the primary and secondary fixations of the acetabular implant component.

Furthermore, once the implant component is implanted and fixed, the convergence of the fixing areas ensures that there are no dead points in the bone anchor (likewise convergent) or in the interference screws. Therefore, based on Wolff's law, the bone around the bone anchor and around the interference screws is prevented from losing bone density as the bone anchor and the interference screws become osseointegrated.

In a second inventive aspect, the invention provides a bone fixation assembly for fixing the prosthetic implant components to the tibia and to the femur of a patient in knee arthroplasties, wherein the assembly comprises:
a bone fixation system with a tibial prosthetic implant component according to the embodiments of the first inventive aspect;
a bone fixation system with a knee femoral prosthetic implant component according to the embodiments of the first inventive aspect; and
a tibiofemoral insert.

This second inventive aspect describes a bone fixation assembly for knee arthroplasties comprising two bone fixation systems, one with a tibial prosthetic implant component and another with a femoral prosthetic implant component, suitable to be implanted in the femoral epiphysis condyle. The assembly further comprises the tibiofemoral insert to which the different prosthetic implant components of the fixation systems would be coupled. In this way, both prosthetic implant components are suitable for being joined to one another by virtue of the coupling of their first faces with the same tibiofemoral insert.

All the advantages explained above for the different embodiments of the bone fixation system with a tibial prosthetic implant component of the first inventive aspect and the advantages explained above for the different embodiments of the bone fixation system with a knee femoral prosthetic implant component of the first inventive aspect can be extrapolated to the second inventive aspect.

In a third inventive aspect, the invention provides a bone fixation assembly for fixing the prosthetic implant components to the femur and to the pelvis of a patient in hip arthroplasties, wherein the assembly comprises:
a bone fixation system with a hip femoral prosthetic implant component according to the embodiments of the first inventive aspect;
a bone fixation system with an acetabular prosthetic implant component according to the embodiments of the first inventive aspect; and
a femoroacetabular insert.

This third inventive aspect describes a bone fixation assembly for hip arthroplasties comprising two bone fixation systems, one with an acetabular prosthetic implant component and another with a femoral prosthetic implant component, suitable to be implanted in the femur. The assembly further comprises a femoroacetabular insert to which the different prosthetic implant components of the fixation systems would be coupled. In this way, both prosthetic implant components are suitable to be joined to one another by virtue of the coupling of their first faces with the same femoroacetabular insert.

All the advantages explained above for the different embodiments of the bone fixation system with a hip femoral prosthetic implant component of the first inventive aspect and the advantages explained above for the different embodiments of the bone fixation system with an acetabular prosthetic implant component of the first inventive aspect can be extrapolated to the third inventive aspect.

In a fourth inventive aspect, the invention provides a bone fixation method for fixing a prosthetic implant component to a bone of a patient in knee or hip arthroplasties, wherein said method comprises the following steps:
(i) providing a bone fixation system according to any of the embodiments of the first inventive aspect,
(ii) carving the bone of the patient to create a bone housing sized and configured to house the bone anchor of the prosthetic implant component of the bone fixation system,
(iii) implanting the prosthetic implant component in the bone by introducing the bone anchor into the bone housing, and
(iv) screwing the plurality of interference screws into the corresponding through conduits of the prosthetic implant component going through the fixing areas.

This fourth inventive aspect describes a bone fixation method for fixing the prosthetic implant component of a fixation system according to any embodiment of the first inventive aspect to a bone of a patient. In particular, the method is carried out during a knee or hip arthroplasty.

In one embodiment, in step (i) a bone fixation system with a tibial prosthetic implant component is provided and a bone housing is created in the tibia of the patient.

In one embodiment, in step (i) a bone fixation system with a knee femoral prosthetic implant component is provided and a bone housing is created in the femoral epiphysis of the patient.

In one embodiment, in step (i) a bone fixation system with a hip femoral prosthetic implant component is provided and a bone housing is created in the femur of the patient.

In one embodiment, in step (i) a bone fixation system with an acetabular prosthetic implant component is provided and a bone housing is created in the pelvis of the patient.

The bone housing must be sized and configured to house the bone anchor of the prosthetic implant component of the bone fixation system. Once said bone housing is carved, the component is implanted in the bone of the patient by introducing the bone anchor into the housing such that, once said bone anchor has been fully inserted, the plurality of interference screws are screwed into the corresponding through conduits of the prosthetic implant component, going through the fixing areas.

The internal emptying of the bone necessary to create the bone housing is such that, once the bone anchor is housed, and/or impacted, and/or inserted, the space between the fixing areas of the through conduits and the bone surrounding same has a dimension equal to or smaller than the inner diameter of the corresponding interference screw.

Throughout the document, the inner diameter of an interference screw shall be understood to mean the diameter of the cylinder and/or cone on which the thread is located, which is measured between roots and also known in the state of the art as the core diameter.

The less the internal emptying is, the greater the synergistic expansive action of the plurality of interference screws which are screwed into the through conduits will be, with support in their corresponding second portions, which increases the primary and secondary fixations required to neutralize the radial and torsional forces impacting the prosthetic implant component once implanted.

All the advantages explained above for the different embodiments of the bone fixation system of the first inventive aspect can be extrapolated to the fourth inventive aspect.

In a particular embodiment, the method comprises the following steps prior to steps (i)-(iv):
a) acquiring at least one image of the bone of the patient, preferably an X-ray, and/or a magnetic resonance image, and/or a computed tomography image, and
b) manufacturing the prosthetic implant component of the fixation system, sizing and personalizing it to the anatomy of the bone of the patient visible in the at least one image such that, when the bone anchor is introduced into the bone of the patient, at least two of the second portions of the through conduits extend in the paracortical direction and/or along the dense trabecular bone of the patient.

In this embodiment, the method further comprises acquiring a medical image of the bone of the patient, for example, an X-ray, a magnetic resonance image, or a computed tomography image, such that the particular morphology of the bone of the patient is known beforehand. In this way, the method continues with a step of manufacturing the prosthetic implant component based on the medical image acquired, wherein the component is personalized so that its shape and dimensions are suitable for the anatomy of the patient in question. In particular, the dimensions and morphology of the prosthetic component must be such that, once the bone anchor is housed in the bone of the patient, two or more of the second portions of the through conduits must extend in the paracortical direction (close to the cortex) and/or along the dense trabecular bone of the patient.

In one embodiment, to perform bone resections, and/or perforations, and/or internal bone emptying required to implant the prosthetic implant component in the bone of the patient, a drilling template personalized in accordance with the previously obtained medical image is used.

In a fifth inventive aspect, the invention provides a bone fixation method for fixing the prosthetic implant components to the bones of a patient in knee or hip arthroplasties, wherein said method comprises the following steps:
(i) providing a bone fixation assembly according to any of the embodiments of the second or third inventive aspects,
(ii) carving the bones of the patient to create bone housings sized and configured to house the bone anchors of the prosthetic implant components of the bone fixation assembly,
(iii) for each bone, implanting the corresponding prosthetic implant component by introducing its bone anchor into the bone housing carved in said bone, and
(iv) screwing the plurality of interference screws into the corresponding through conduits of the prosthetic implant components going through the fixing areas.

All the features and/or steps of the methods described in this specification (including the claims, description, and drawings) can be combined in any combination, with the exception of combinations of such mutually exclusive features.

### DESCRIPTION OF THE DRAWINGS

These and other features and advantages of the invention will become more apparent based on the following detailed description of a preferred embodiment, given only by way of illustrative and non-limiting example in reference to the attached figures.

FIGS. 1A-1B These figures show different views of a bone fixation system for fixing a prosthetic implant component for knee arthroplasties of the state of the art.

FIGS. 2A-2E These figures illustrate different views and details of a bone fixation system for fixing a prosthetic implant component according to an embodiment of the invention, wherein the prosthetic implant component is a knee tibial prosthetic implant component.

FIGS. 3A-3E These figures illustrate different views and details of a bone fixation system for fixing a prosthetic implant component according to an embodiment of the invention, wherein the prosthetic implant component is a knee femoral prosthetic implant component.

FIG. 4 This figure shows a bone fixation assembly for fixing the components of a knee prosthetic implant assembly to the tibia and to the femur of a patient according to an embodiment of the invention.

FIGS. 5A-5D These figures illustrate different views and details of a bone fixation system for fixing a prosthetic implant component according to an embodiment of the invention, wherein the prosthetic implant component is a hip femoral prosthetic implant component.

FIGS. 6A-6D These figures illustrate different views and details of a bone fixation system for fixing a prosthetic implant component according to an embodiment of the invention, wherein the prosthetic implant component is an acetabular prosthetic implant component.

FIG. 7 This figure shows a bone fixation assembly for fixing the components of a hip prosthetic implant assembly to the femur and to the pelvis of a patient according to an embodiment of the invention.

FIGS. 8A-8B These figures show diagrams of a bone fixation method for fixing a prosthetic implant component to a bone of a patient according to two embodiments of the invention.

### DETAILED DESCRIPTION OF THE INVENTION

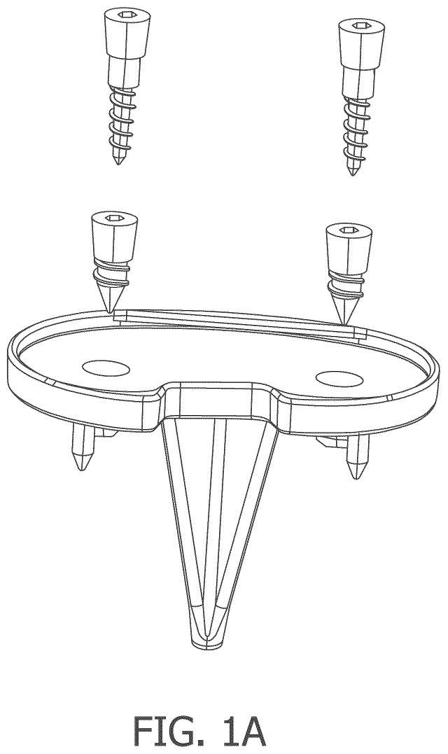

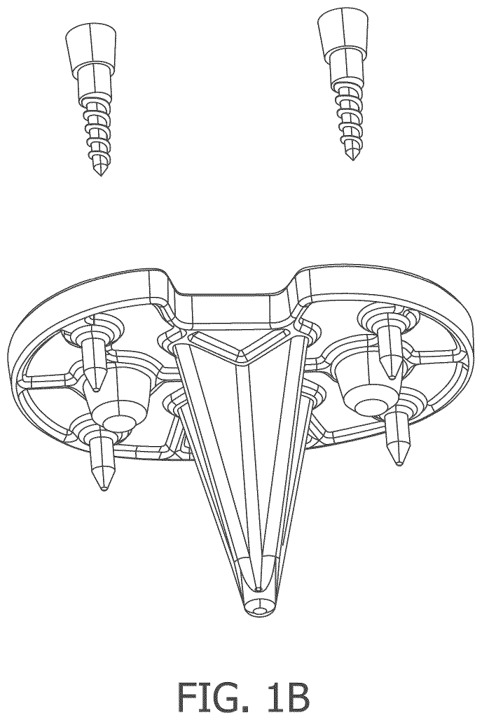

FIGS. 1A-1B show different views of a bone fixation system for fixing a prosthetic implant component for knee arthroplasties of the state of the art, the prosthetic implant component being particularly a tibial prosthetic implant component.

As can be seen in both figures, the system comprises a prosthetic implant component formed by a prosthetic body and a bone anchor, and axial fixation screws comprising a first longitudinal portion, intended to be screwed completely into the prosthetic implant component, and a second longitudinal portion, intended to be screwed completely into the bone of the patient.

As discussed above, this fixation system by means of solely axial fixation screws has two significant problems associated therewith: onset of osteolysis around the screws and alteration of the original force lines of the bone.

As can be seen in FIGS. 1A and 1B, the screws are screwed in exclusively perpendicular to the prosthetic body, so they do not interact with the bone anchor, and therefore do not create a fixing area in said bone anchor. This configuration provides a maximum primary axial fixation force and high mechanical fixation.

However, with this configuration, bone density loss occurs around the screws, that is, osteolysis occurs around said screws, as the prosthetic implant component becomes osseointegrated.

Furthermore, with this configuration, the original force lines of the bone are altered. Bone morphology determines the force lines and vice versa, so bone morphology is altered if the force lines are altered, with this alteration being one of the most common causes of periprosthetic fractures.

The other figures show a bone fixation system (1), a bone fixation assembly (3) and a bone fixation method according to different embodiments of the invention.

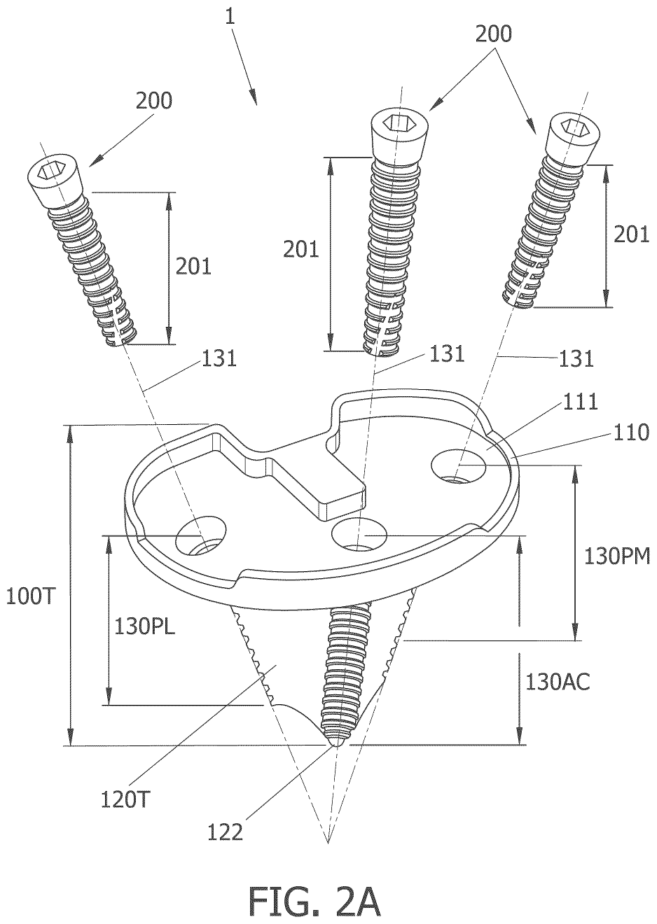

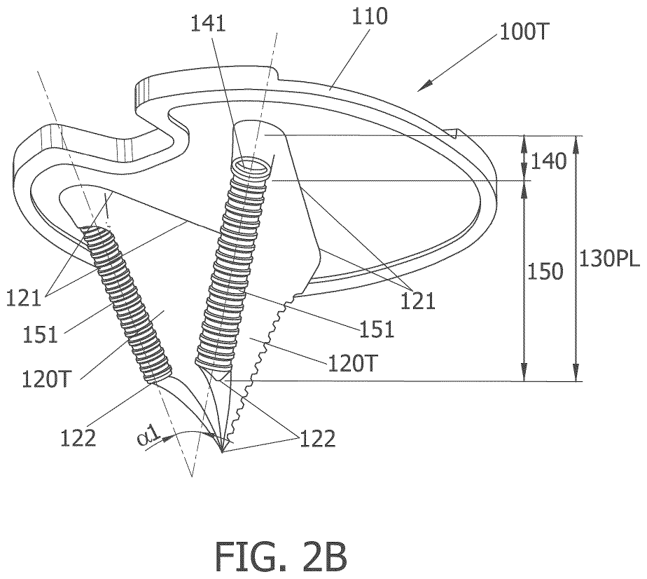

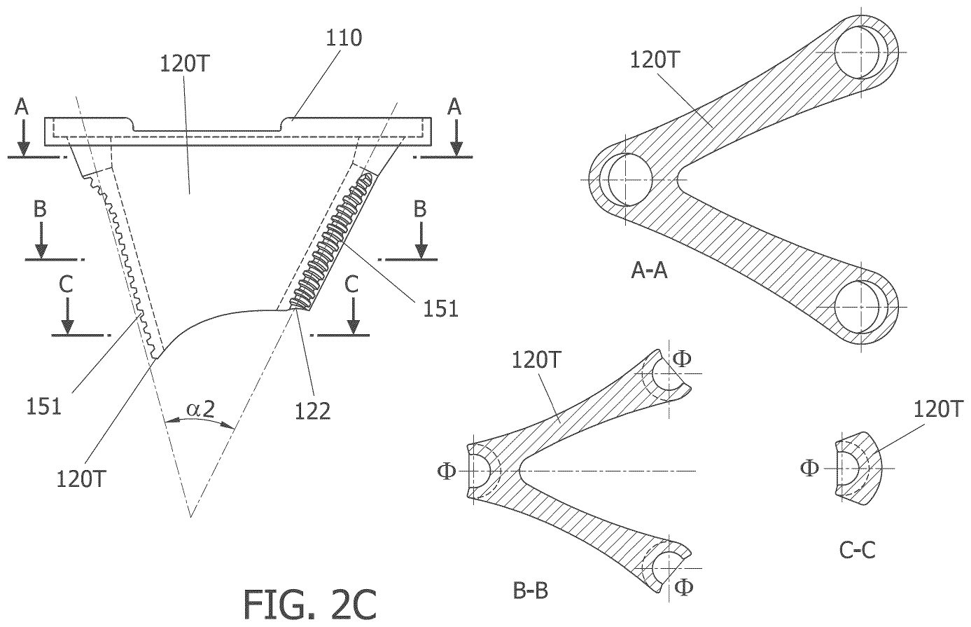

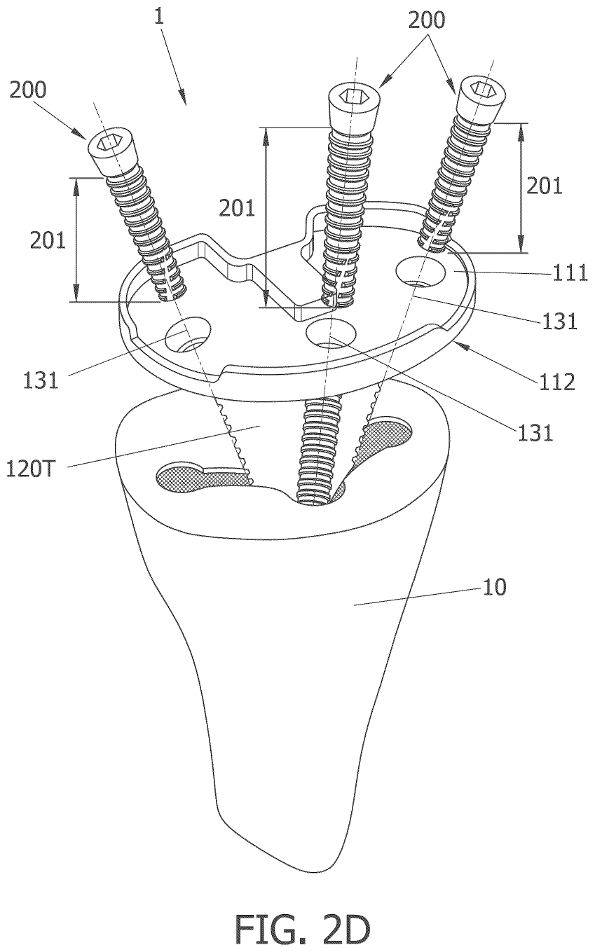

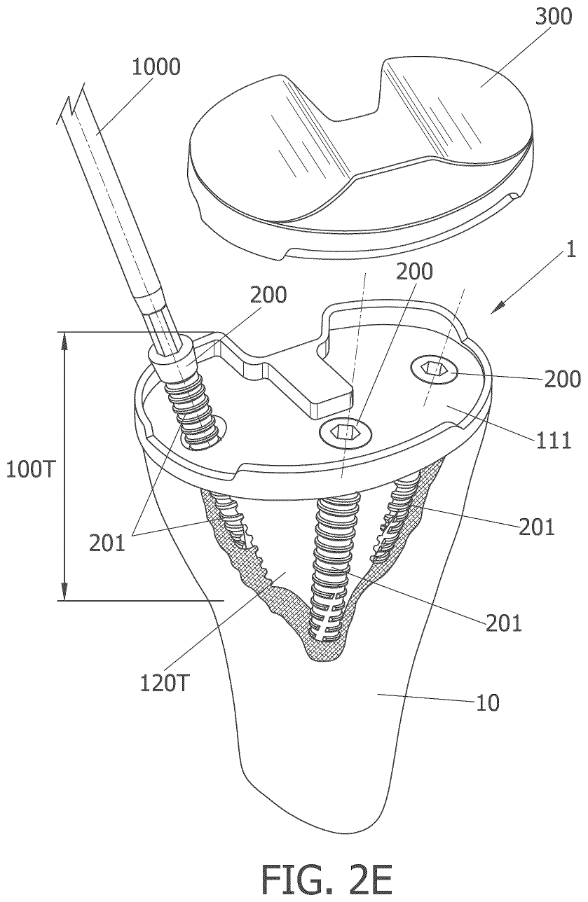

FIGS. 2A-2E, 3A-3E, 5A-5D, and 6A-6D show different embodiments of a bone fixation system (1) for fixing a prosthetic implant component (100) to a bone (10) of a patient in knee or hip arthroplasties, wherein said system (1) comprises:
a prosthetic implant component (100) comprising:
   a prosthetic body (110) comprising a first face (111), configured to be coupled at least partially to a prosthetic insert (300, 400), and a second face (112), configured to at least partially contact with the bone (10),
   a bone anchor (120), configured to be implanted in the bone (10), comprising a proximal end (121), attached to the second face (112) of the prosthetic body (110), and a distal end (122), and
   a plurality of through conduits (130), each of which is oriented according to a longitudinal axis (131), and each of which comprising:
      a first portion (140), formed as a closed conduit in the prosthetic body (110) going through said prosthetic body (110) from the first face (111) to the second face (112) and, optionally, partially going through the bone anchor (120), and
      a second portion (150), after the first portion (140), which is formed as an open conduit in the bone anchor (120) defining a fixing area (160),
   wherein:
   the first portion (140) and/or the second portion (150) comprise a first threading region (141, 151), and
   the longitudinal axes (131) of at least two of the plurality of through conduits (130) form an angle greater than zero degrees and less than ninety degrees, the angle being closed in the proximal-distal direction,
a plurality of interference screws (200), each of which
   comprises a second threading region (201), complementary to the first threading region (141, 151) of a corresponding through conduit (130), and
   is configured to be screwed into a corresponding through conduit (130), and
      wherein, when the bone anchor (120) is implanted in a bone housing carved in the bone (10), the prosthetic implant component (100) is fixed to the bone (10) when the interference screws (200) are screwed into the through conduits (130) going through the fixing areas (160).

More particularly, the angle formed by the longitudinal axes (131) of at least two through conduits (130) of the plurality of through conduits (130) is in the range of 5-65 degrees.

Advantageously, the through conduits and their corresponding fixing areas are osteomorphically arranged, i.e., arranged depending on the particular anatomy and on the force line distribution of each particular bone.

In one embodiment, the second portion (150) of each through conduit (130) of the plurality of through conduits (130) is formed as an open conduit the cross-section of which is in the shape of a circumference arch with an angle Φ equal to or greater than 180°.

Advantageously, the circumference arch of the cross-section of the conduit with an angle equal to or greater than 180°, attached to the convergence of the fixing areas in the proximal-distal direction, ensures that there are no dead points, i.e., points that do not receive load, around the bone anchor or around the interference screws when the patient is standing or walking.

In this way, as discussed above, based on Wolff's law, the bone around the bone anchor, around the prosthetic implant component, and around the interference screws is prevented from losing bone density as they both become osseointegrated.

In one embodiment, the plurality of interference screws (200) comprise a metric of between 6 mm and 20 mm, and a length of between 15 mm and 100 mm.

In these figures, the interference screws (200) are cylindrical, cannulated, with a blunt trapezoidal double thread, an inverted frustoconical head, and a rounded tip.

Advantageously, the long and wide trapezoidal double thread provides improved grip on the bone (10), while the hollow central shaft provides improved torque transmission and facilitates the screwing in of the interference screw (200).

Advantageously, the blunt thread protects the bone cortex when, once the prosthetic implant component (100) is implanted, the interference screws (200) are screwed into the dense trabecular bone (10), between each of the second portions (150) of the through conduits (130) and the cortical bone (10).

In other embodiments, the interference screws (200) have different features. For example, the thread of said interference screws (200) is squared, rounded, or trapezoidal.

In these figures, furthermore:
the first portion (140) of each through conduit (130) comprises a proximal part (142) and a distal part (143), wherein
   the distal part (143) comprises the first threading region (141), and
   the proximal part (142) has a frustoconical section; and
each interference screw (200) comprises a frustoconical head complementary to the proximal part of the first portion (140).

In these embodiments, the system (1) optionally comprises:
a screwdriver element (1000) configured to screw the interference screws (200) to the prosthetic implant component (100), and/or
a bone preparation element configured to carve the bone (10) and create a bone housing sized and configured to house the bone anchor (120).

FIG. 2A shows an exploded top view of the bone fixation system (1) according to an embodiment of the invention, wherein the prosthetic implant component (100) is a tibial prosthetic implant component (100T), and wherein:
the first face (111) of the prosthetic body (110T) is configured to be coupled at least partially to a tibiofemoral insert (300) and the second face (112) is configured to contact a tibial bone resection plane,
the bone anchor (120T) comprises an anterocentral tibial piece, a posteromedial tibial piece, and a posterolateral tibial piece attached to one another, and
the plurality of through conduits (130) comprises an anterocentral through conduit (130AC), a posteromedial through conduit (130PM), and a posterolateral through conduit (130PL), and
   wherein
the longitudinal axes (131) of the posterolateral through conduit (130PL) and the posteromedial through conduit (130PM) form an angle α₁ of 45°,
the longitudinal axis (131) of the anterocentral through conduit (130AC) forms, with each of the longitudinal axes (131) of the posterolateral through conduit (130PL) and the posteromedial through conduit (130PM), an angle α₂ of 20 degrees, and
   wherein the bone fixation system (1) comprises three interference screws (200).

Advantageously, an angle α₁ of 45° closed in the proximal-distal direction is similar to the anatomical angle defined between the interosseous and medial edges of the proximal tibial epiphysis, and an angle α₂ of 30° is similar to the anatomical angles defined between the anterior edge and the interosseous and medial edges of the proximal tibial epiphysis, so this design of a tibial prosthetic implant component (100T) preserves three of the main physiological load distribution lines of the proximal tibial epiphysis.

Advantageously, the three interference screws (200) work together to provide a synergistic effect whereby each interference screw (200), in addition to fixing the prosthetic implant component (100T) in the fixing area (160) thereof, by means of axial force, provides an expansive force which is transmitted through the bone anchor (120T) to the area of the other two interference screws (200). This synergistic expansive action of the three interference screws (200) would not occur in prosthetic implant components (200) with a single interference screw; for example, with a single anterocentral interference screw.

In other embodiments, the longitudinal axes (131) of the posterolateral through conduit (130PL) and the posteromedial through conduit (130PM) form an angle α₁ between 30 and 65 degrees.

In other embodiments, the longitudinal axis (131) of the anterocentral through conduit (130AC) forms, with each of the longitudinal axes (131) of the posterolateral through conduit (130PL) and the posteromedial through conduit (130PM), an angle α₂ between 15 and 45 degrees.

In one embodiment, the anterocentral tibial piece, the posteromedial tibial piece, and the posterolateral tibial piece are not attached to one another.

In one embodiment, each of the pieces of the bone anchor (120T) is formed as a pyramid frustum or a cone frustum.

In the embodiment shown in FIGS. 2A and 2B, which are top and rear bottom views, respectively, the anterocentral tibial piece has a length greater than the length of the posteromedial and posterolateral tibial pieces, and, in return, the anterocentral interference screw has a length greater than the length of the posteromedial and posterolateral interference screws. In other embodiments, the lengths of the tibial pieces and/or of the interference screws are different.

In the embodiment shown in FIG. 2A, the posteromedial and posterolateral interference screws (200) have a diameter of 9 mm and a length of 30 mm, whereas the anterocentral interference screw has a diameter of 12 mm and a length of 40 mm. The corresponding through conduits (130) comprise reciprocal dimensions. In other embodiments, the dimensions are different.

FIG. 2C shows different cross-sections of the bone anchor (120T) of this embodiment of the bone fixation system (1), the sections taken at different heights of the bone anchor (120T).

In particular, section A-A represents a proximal cross-section (close to the prosthetic body (110)), section B-B represents a medial cross-section (halfway between the two ends of the bone anchor (120T)), and cross-section C-C represents a distal cross-section (close to the end first introduced into the bone cavity of the tibia). The thickness of said bone anchor (120T) therefore decreases in the proximal-distal direction.

As can be seen in this figure, cross-sections B-B and C-C go through the second portions (150) of one or more through conduits (130) of the plurality of through conduits (130), with said second portions (150) being formed as open conduits, the cross-section of which is in the shape of a circumference arch with an angle Φ equal to 180°. In other embodiments, the angle Φ is greater than 180°.

FIG. 2D shows the same bone fixation system (1) as the one illustrated in FIGS. 2A-2C, but being inserted into the tibia (10), specifically into a bone housing carved in this bone (10) of the patient for such purpose.

In FIG. 2E, the same bone fixation system (1) is implanted in the bone (10) of the patient and the interference screws (200) are screwed into the through conduits (130).

The bone fixation system (1) optionally comprises a screwdriver element (1000) such as that shown in FIG. 2E. This screwdriver element (1000) is configured to screw the interference screws (200) to the prosthetic implant component (100). This figure also shows the tibiofemoral insert (300) to which the first face (111) of the prosthetic body (110T) can be at least partially coupled, said insert (300) not being part of the bone fixation system (1).

In other embodiments, additionally or alternatively, the bone fixation system (1) comprises a bone preparation element configured to carve the bone (10) and create a bone housing sized and configured to house the bone anchor (120T).

The embodiments shown in FIGS. 2A-2E are suitable for fixing tibial prosthetic implant components (100T) for knee arthroplasties, preserving the posterior cruciate ligament.

In other embodiments, the fixation system of the invention is suitable for fixing tibial prosthetic implant components (100T) of the “posterior stabilized”, “ultra-congruent”, and/or “rotating platform” types, as well as for fixing unicompartmental tibial prosthetic implant components (100T).

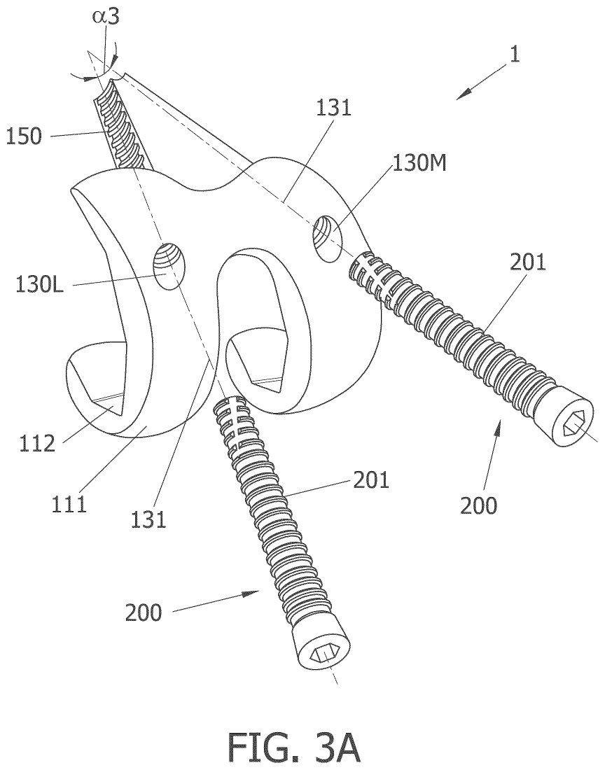

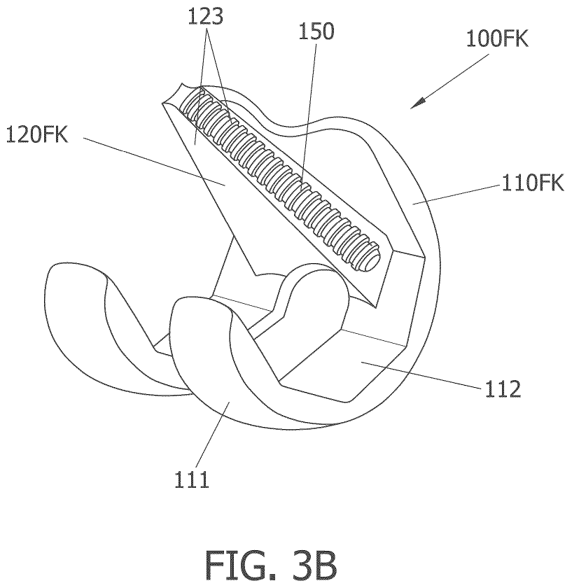

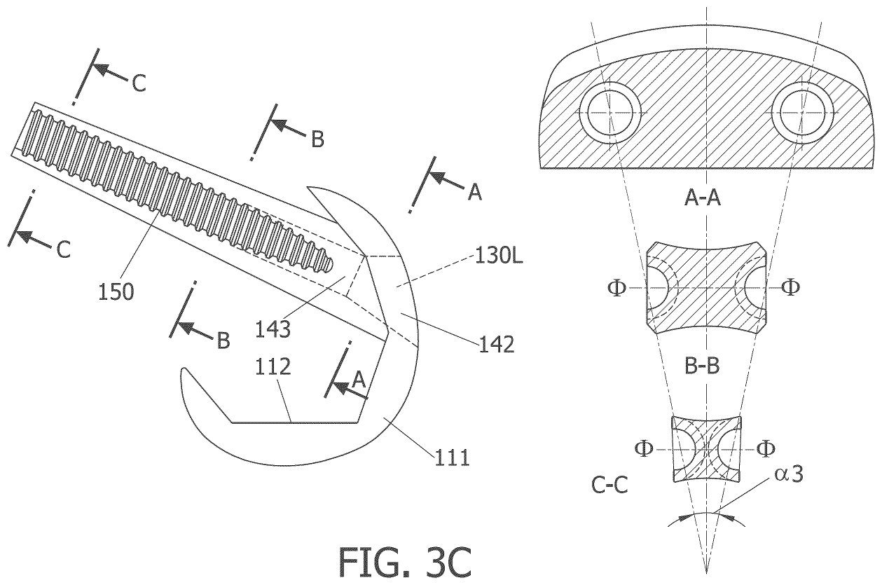

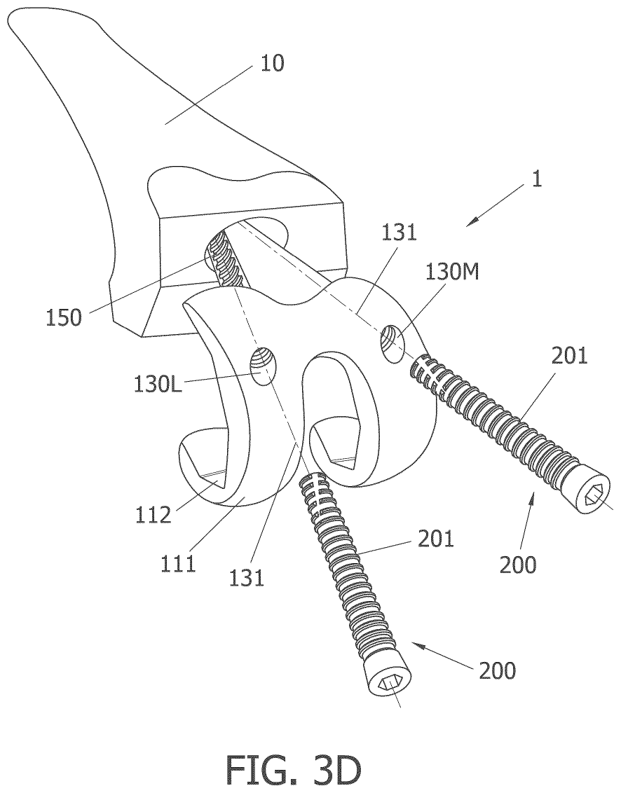

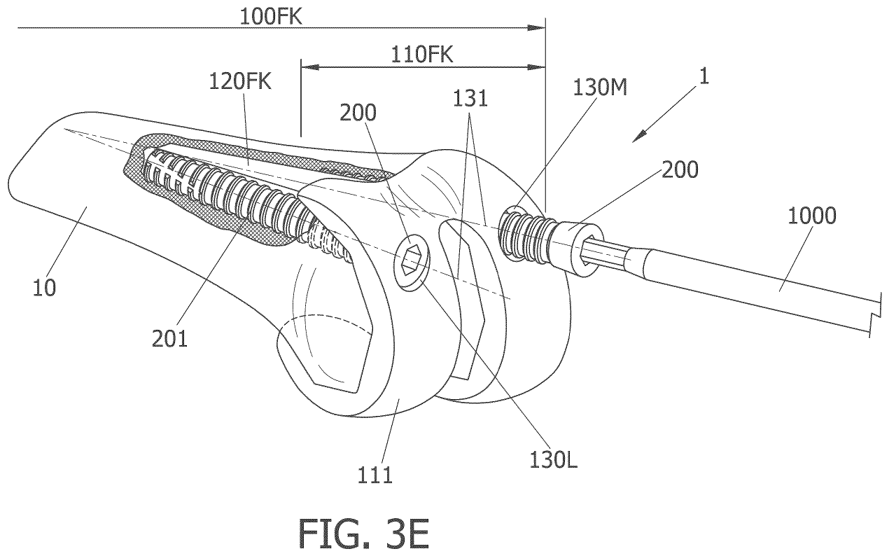

FIG. 3A shows an exploded front view of the bone fixation system (1) according to an embodiment of the invention, wherein the prosthetic implant component (100) is a knee femoral prosthetic implant component (100FK) and:
the first face (111) of the prosthetic body (110FK) is configured to be coupled at least partially to a tibiofemoral insert (300) and the second face (112) is configured to contact a femoral condyle bone resection plane,
the plurality of through conduits (130) comprises a medial through conduit (130M) and a lateral through conduit (130L), and
   wherein the longitudinal axes (131) of the medial through conduit (130M) and of the lateral through conduit (130L) form an angle α₃ of 30 degrees, and wherein the bone fixation system (1) comprises two interference screws (200).

Furthermore, FIG. 3A illustrated that the bone anchor (120FK) comprises a femoral piece formed as a pyramid frustum which, with the exception of its base, comprises four concave faces (123).

Advantageously, an angle α₃ of 30 degrees closed in the proximal-distal direction is similar to the anatomical angle defined between the medial and lateral supracondylar lines which delimit, laterally and medially, respectively, the popliteal face of the femur, so this design of the knee femoral prosthetic implant component (100FK) preserves two of the main physiological load distribution lines of the femoral epiphysis of the femur.

Advantageously, the medial and lateral locations of both interference screws (200) provide a synergistic effect whereby, each interference screw (200), in addition to fixing the prosthetic implant component (100FK) in the fixing area (160) thereof, by means of axial force, provides an expansive force which is transmitted through the bone anchor (120FK) to the area of the other interference screw (200). This synergistic expansive action of the two interference screws (200) reinforces the primary and secondary fixations of the prosthetic implant component (100FK), preventing micromovements which may cause the loosening thereof and the subsequent arthroplasty failure due to this reason.

The interference screws (200) of this embodiment have a diameter of 9 mm and a length of 30 mm. Both through conduits (130) comprise reciprocal dimensions. In other embodiments, the dimensions are different.

In other embodiments, the longitudinal axes (131) of the medial through conduit (130M) and of the lateral through conduit (130L) form an angle α₃ between 15 and 45 degrees.

FIG. 3B is a rear side view of the prosthetic implant component (100FK) of FIG. 3A.

FIG. 3C shows different cross-sections of the bone anchor (120FK) of this embodiment of the bone fixation system (1), the sections taken at different heights of the bone anchor (120).

In particular, section A-A represents a proximal cross-section (close to the prosthetic body (110)), section B-B represents a medial cross-section (halfway between the two ends of the bone anchor (120FK)), and cross-section C-C represents a distal cross-section (close to the end first introduced into the bone cavity of the tibia). The thickness of said bone anchor (120FK) therefore decreases in the proximal-distal direction.

As can be seen in this figure, cross-sections B-B and C-C go through the second portions (150) of the through conduits (130), said second portions (150) being formed as open conduits, the cross-section of which is in the shape of a circumference arch with an angle Φ equal to 180°. In other embodiments, the angle Φ is greater than 180°.

FIG. 3D shows the same bone fixation system (1) as the one shown in FIGS. 3A-3C, but being inserted into the femur (10), specifically into a bone housing carved in this bone (10) of the patient for such purpose.

In FIG. 3E, the bone fixation system (1) is implanted in the bone (10) of the patient and the interference screws (200) are screwed into the through conduits (130).

The bone fixation system (1) optionally comprises a screwdriver element (1000) such as that shown in FIG. 3E. This screwdriver element (1000) is configured to screw the interference screws (200) to the prosthetic implant component (100FK). This figure also shows the tibiofemoral insert (300) to which the first face (111) of the prosthetic body (110FK) can be at least partially coupled, said insert (300) not being part of the bone fixation system (1).

In other embodiments, additionally or alternatively, the bone fixation system (1) comprises a bone preparation element configured to carve the bone (10) and create a bone housing sized and configured to house the bone anchor (120FK).

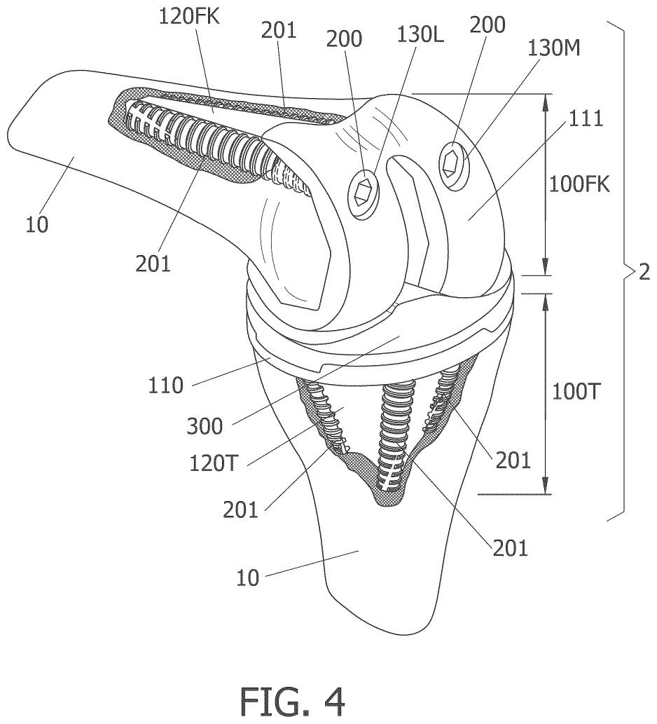

FIG. 4 shows a bone fixation assembly (2) for fixing the prosthetic implant components (100) to the tibia (10) and to the femur (10) of a patient in knee arthroplasties, wherein the assembly (2) comprises:
a bone fixation system (1) with a tibial prosthetic implant component (100T) shown in FIGS. 2A-2E;
a bone fixation system (1) with a knee femoral prosthetic implant component (100FK) such as that shown in FIGS. 3A-3E; and
a tibiofemoral insert (300).

Advantageously, the bone fixation assembly (2) allows preserving the original physiological load distribution of the knee, providing a gradual transmission of forces, directly through the corresponding prosthetic implant components (100T, 100FK), and indirectly through the corresponding interference screws (200), which:
increases the resistance of the interference screws (200) to bending and torsional loads which may cause micromovements and possible reabsorption of bone (10) around said screws; and
preserves the bone stock, preventing hyprepressure regions, which favors functional bone regeneration, in order to prevent periprosthetic fractures.

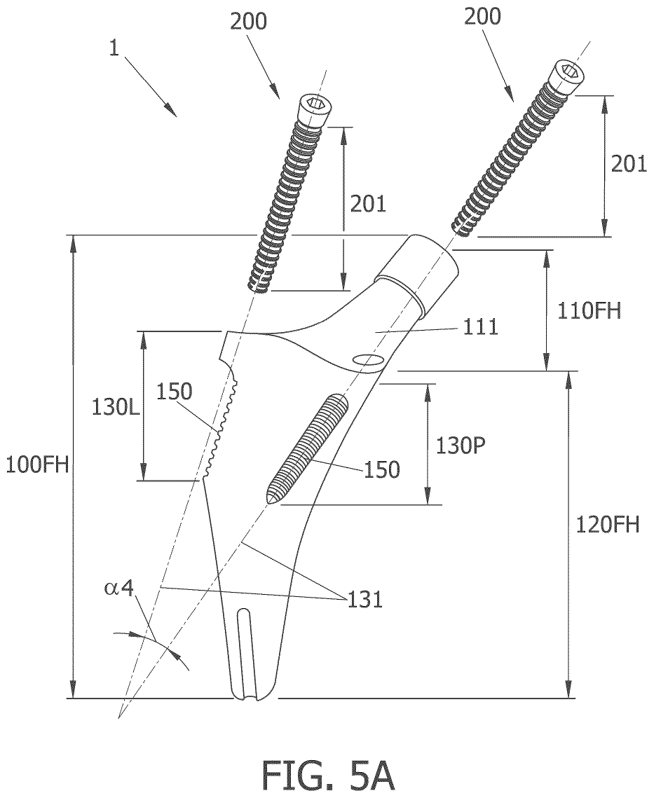

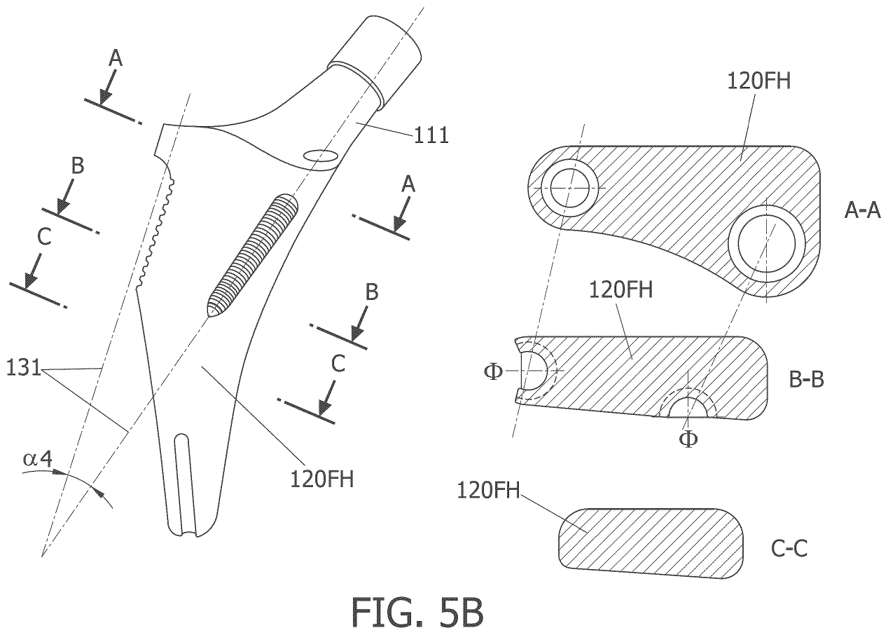

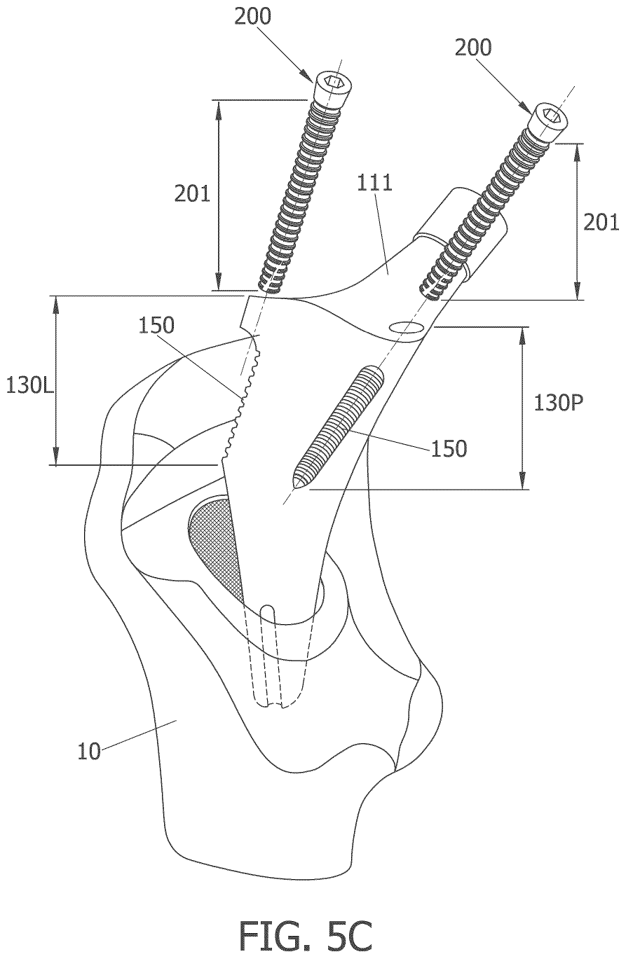

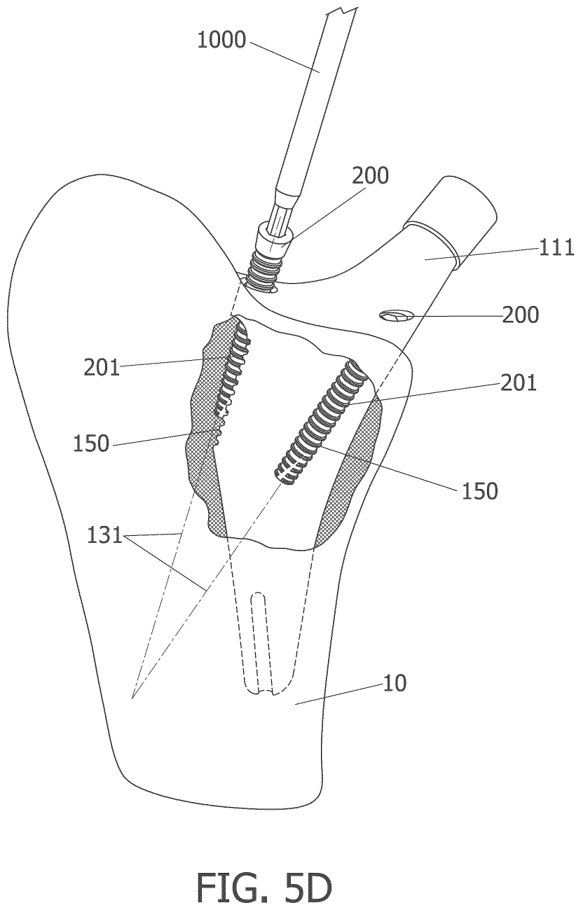

FIG. 5A shows an exploded rear view of the bone fixation system (1) according to an embodiment of the invention, wherein the prosthetic implant component (100) is a hip femoral prosthetic implant component (100FH), and:
the second face (112) is configured to contact a femoral epiphysis bone resection plane,
the first face (111) of the prosthetic body (110FH) is configured to be coupled at least partially to a femoroacetabular insert (400),
the plurality of through conduits (130) comprises a lateral through conduit (130L) and a posterior through conduit (130P), and
   wherein the longitudinal axes (131) of the lateral through conduit (130L) and of the posterior through conduit (130P) form an angle α₄ of 30 degrees, and wherein the bone fixation system (1) comprises two interference screws (200).

Advantageously, an angle α₄ of 30° closed in the proximal-distal direction is similar to the anatomical angle defined between the line tangent to the femoral neck and the line following the profile of the calcar, so this design of the hip femoral prosthetic implant component (100FH) preserves two of the main physiological load distribution lines of the femoral epiphysis.

Advantageously, once screwed in, both interference screws (200) provide a double fixation of the hip femoral prosthetic implant component (100FH). On one hand, each interference screw (200) fixes the hip femoral prosthetic implant component (100FH) in the fixing area (160) thereof, by means of axial force and, on the other hand, each interference screw (200) provides an expansive force that retains the prosthetic implant component (100FH) against the dense trabecular bone of the opposite fixing area.

Advantageously, the fixation system (1) of this embodiment allows the bone anchor (120FH) to have a short, curved wedge design. This promotes a physiological load distribution which reduces the risk of periprosthetic fracture, while at the same time facilitating soft tissue preservation techniques and preventing thigh pain associated with hip implant components having a long shank.

The interference screws (200) of this embodiment have a diameter of 9 mm and a length of 40 mm. Both through conduits (130) comprise reciprocal dimensions. In other embodiments, the dimensions are different.

In other embodiments, the longitudinal axes (131) of the lateral through conduit (130L) and of the posterior through conduit (130P) form an angle α₄ between 5 and 45 degrees.

FIG. 5B shows different cross-sections of the bone anchor (120FH) of this embodiment of the bone fixation system (1), the sections taken at different heights of the bone anchor (120FH).

In particular, section A-A represents a proximal cross-section (close to the prosthetic body (110FH)), section B-B represents a medial cross-section (halfway between the two ends of the bone anchor (120FH)), and cross-section C-C represents a distal cross-section (close to the end first introduced into the bone cavity of the femur). The thickness of said bone anchor (120FH) therefore decreases in the proximal-distal direction.

As can be seen in this figure, cross-section B-B goes through the second portions (150) of the through conduits (130), said second portions (150) being formed as open conduits, the cross-section of which is in the shape of a circumference arch with an angle Φ equal to 180°. In other embodiments, the angle Φ is greater than 180°.

FIG. 5C shows the same bone fixation system (1) as the one shown in FIGS. 5A-5B, but being inserted into the femur (10), specifically into a bone cavity carved in this bone (10) of the patient for such purpose.

In FIG. 5D, the bone fixation system (1) is implanted in the bone (10) of the patient and the interference screws (200) are screwed into the through conduits (130).

The system (1) optionally comprises a screwdriver element (1000) such as that shown in FIG. 5D. This screwdriver element (1000) is configured to screw the interference screws (200) to the prosthetic implant component (100FH).

In other embodiments, additionally or alternatively, the bone fixation system (1) comprises a bone preparation element configured to carve the bone (10) and create a bone housing sized and configured to house the bone anchor (120FH).

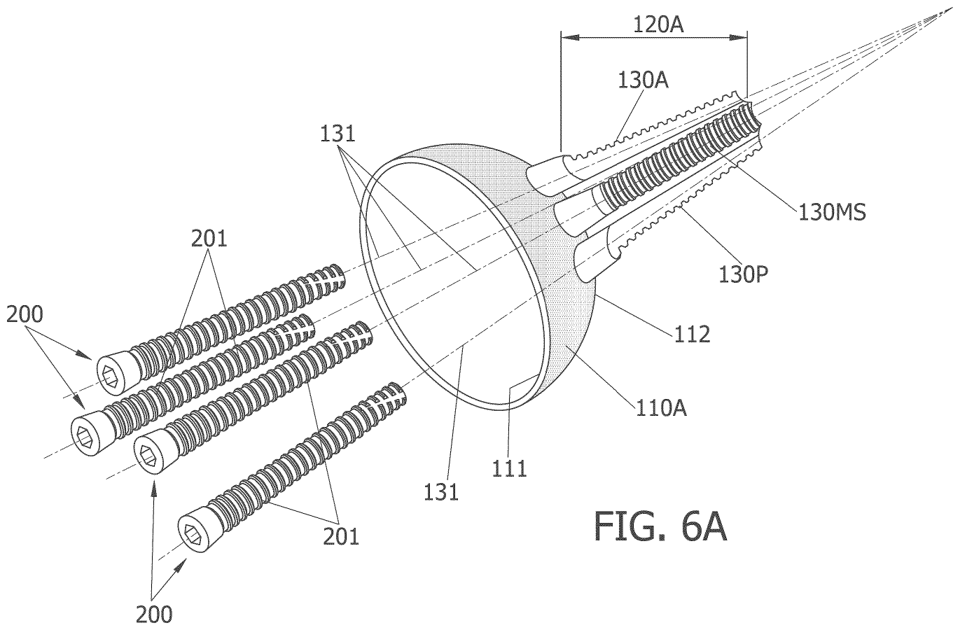

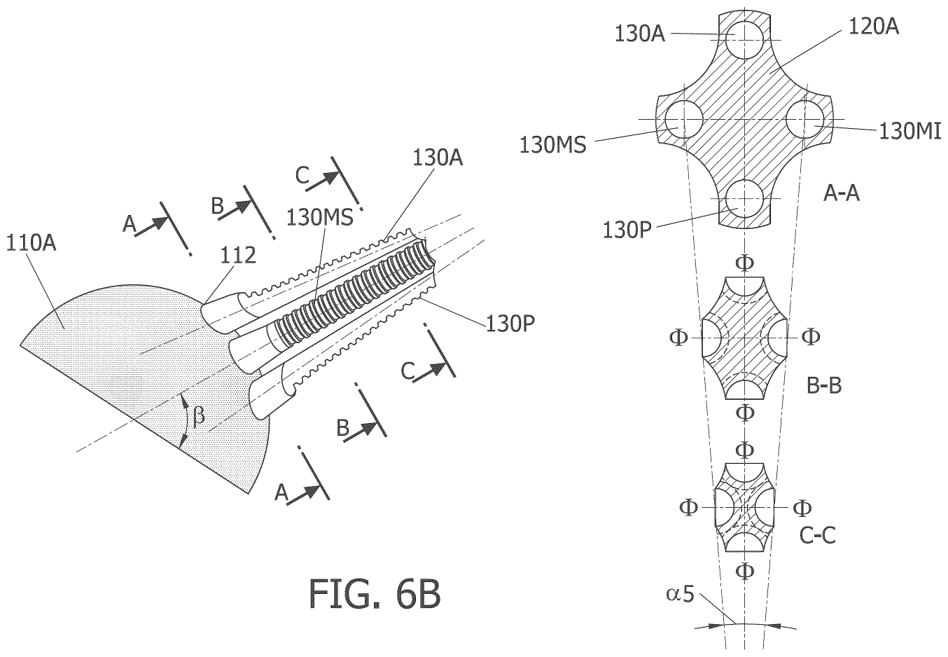

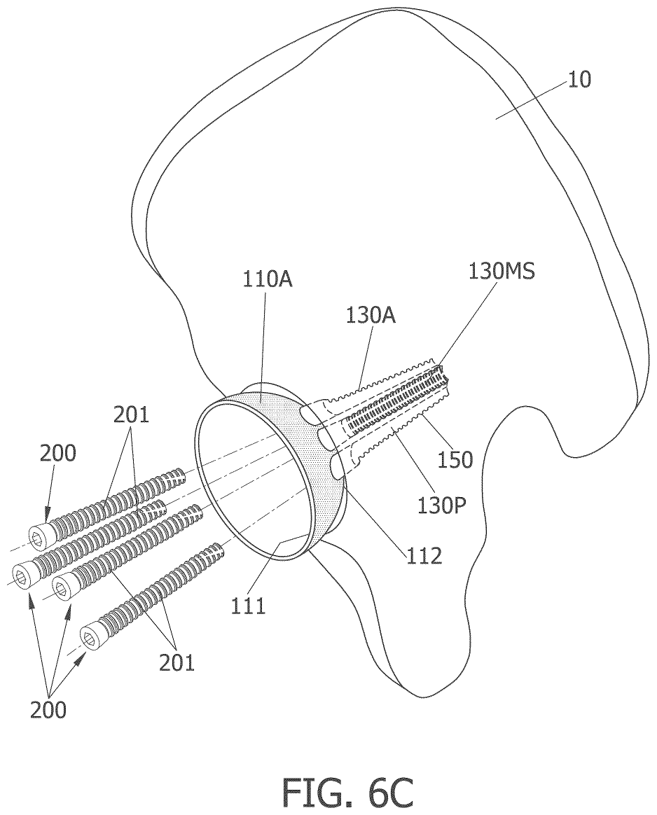

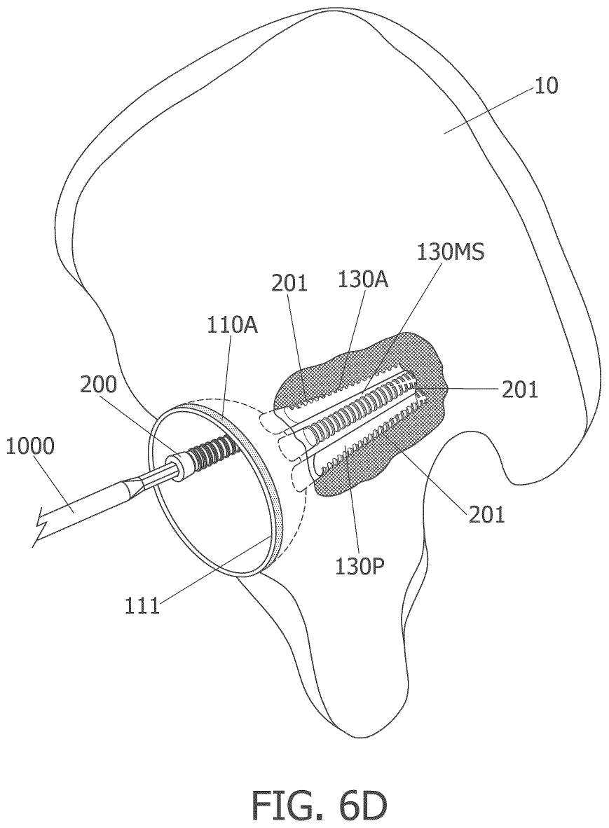

FIG. 6A shows an exploded rear view of the bone fixation system (1) according to an embodiment of the invention, wherein the prosthetic implant component (100) is an acetabular prosthetic implant component (100A), and:
the prosthetic body (110) is an acetabular prosthetic body (110A) formed as an acetabular cup comprising a circumference flange comprised in plane π,
the second face (112) of the acetabular prosthetic body (110A) is configured to contact the acetabular bone (10),
the first face (111) of the acetabular prosthetic body (110A) is configured to be coupled at least partially to a femoroacetabular insert (400),
the plurality of through conduits (130) comprises an anterior through conduit (130A), a medial-superior through conduit (130MS), a medial-inferior through conduit (130MI), and a posterior through conduit (130P), and
   wherein
the longitudinal axes (131) of the medial-inferior through conduit (130MI) and the medial-superior through conduit (130MS) form an angle α₅ of 30 degrees,
the longitudinal axis (131) of the medial-superior through conduit (130MS) forms an angle β of 60 degrees with respect to plane π, and
   wherein the bone fixation system (1) comprises four interference screws (200).

FIGS. 6A and 6B illustrate that the bone anchor (120A) comprises a piece formed as a pyramid frustum inclined with respect to plane π, which comprises in its medial cross-section eight concave faces (123).
FIG. 6B shows different cross-sections of the bone anchor (120A) of this embodiment of the bone fixation system (1), the sections taken at different heights of the bone anchor (120A).

In particular, section A-A represents a proximal cross-section (close to the prosthetic body (110A)), section B-B represents a medial cross-section (halfway between the two ends of the bone anchor (120A)), and cross-section C-C represents a distal cross-section (close to the end first introduced into the bone cavity of the ilium). The thickness of said bone anchor (120A) therefore decreases in the proximal-distal direction.

As can be seen in this figure, cross-sections B-B and C-C go through the second portions (150) of one or more through conduits (130) of the plurality of through conduits (130), said second portions (150) being formed as open conduits, the cross-section of which is in the shape of a circumference arch with an angle Φ equal to 180°. In other embodiments, the angle Φ is greater than 180°.

Advantageously, an angle α₅ of 30° closed in the proximal-distal direction is similar to the anatomical angle defined between the medial and lateral faces of the ilium body, while an angle β of 60° provides a longitudinal axis of the medial-superior through conduit (130MS) which coincides with the insertion axis of the acetabular prosthetic implant component (100A), all of which allows the four fixing areas (160) to be placed parallel, in twos, to both faces of the ilium body, along the dense subcortical trabecular bone, thereby preserving the original physiological load distribution of the hip.

Advantageously, the synergistic action of the four interference screws (200) prevents micromovements from occurring in the prosthetic implant component (100A), and therefore prevents the aseptic loosening of previously osseointegrated acetabular implant components, which currently represents a major problem associated with the acetabular uncemented prosthetic implant components of the state of the art.

In this embodiment, the medial-superior, medial-inferior, and posterior interference screws (200) have a diameter of 9 mm and a length of 30 mm, whereas the anterior interference screw likewise comprises a diameter of 9 mm and a length of 40 mm. The corresponding through conduits (130) comprise reciprocal dimensions. In other embodiments, the dimensions are different.

In other embodiments, the longitudinal axes (131) of the medial-inferior through conduit (130MI) and the medial-superior through conduit (130MS) form an angle α₅ between 15 and 60 degrees.

In other embodiments, the longitudinal axis (131) of the medial-superior through conduit (130MS) forms an angle β between 30 and 90 degrees with respect to plane π.

FIG. 6C shows the same bone fixation system (1) as the one shown in FIGS. 6A-6B, but being inserted into the pelvis, specifically into a bone housing carved in the ilium (10) of the patient for such purpose.

In FIG. 6D, the bone fixation system (1) is implanted in the bone (10) of the patient and the interference screws (200) are screwed into the through conduits (130).

The system (1) optionally comprises a screwdriver element (1000) such as that shown in FIG. 6D. This screwdriver element (1000) is configured to screw the interference screws (200) to the acetabular prosthetic implant component (100A).

In other embodiments, additionally or alternatively, the bone fixation system (1) comprises a bone preparation element configured to carve the bone (10) and create a bone housing sized and configured to house the bone anchor (120).

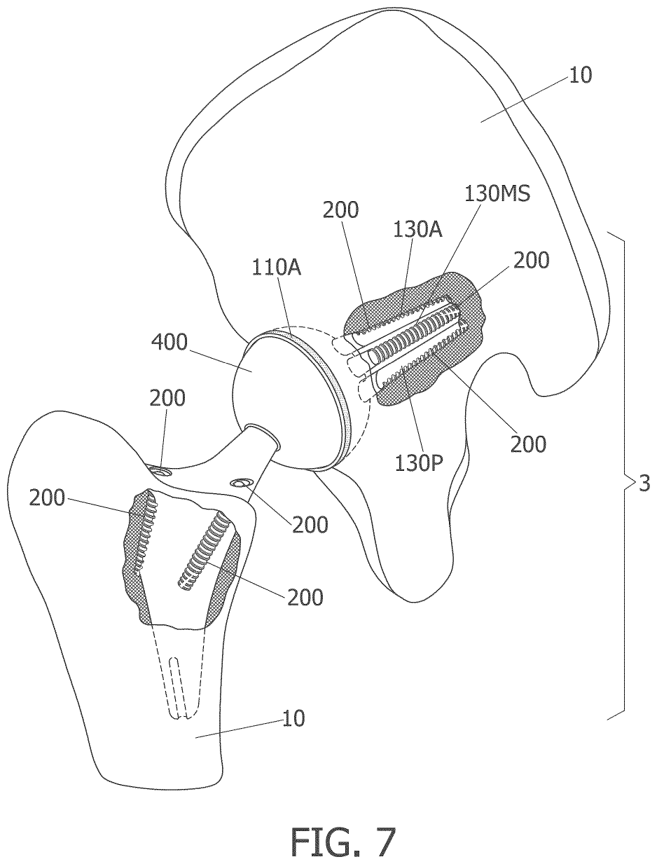

FIG. 7 shows a bone fixation assembly (3) for fixing the prosthetic implant components (100) to the femur (10) and to the pelvis (10) of a patient in hip arthroplasties, wherein the assembly (3) comprises:
a bone fixation system (1) with a hip femoral prosthetic implant component (100FH) shown in FIGS. 5A-5D;
a bone fixation system (1) with an acetabular prosthetic implant component (100A) such as that shown in FIGS. 6A-6D; and
a femoroacetabular insert (400).

This figure shows the femoroacetabular insert (400) to which the first faces (111) of the prosthetic bodies (110A, 110FH) of the bone fixation systems (1) of the bone fixation assembly (3) are at least partially coupled. Said femoroacetabular insert (400) is part of the bone fixation assembly (3), but not part of the bone fixation system (1) with a hip femoral prosthetic implant component (100FH) or part of the bone fixation system (1) with an acetabular prosthetic implant component (100A).

The bone fixation assembly (3) of this embodiment allows preserving the original physiological load distribution of the hip, providing a gradual transmission of forces, directly through the corresponding prosthetic implant component (100), and indirectly through the corresponding interference screws (200), which:
increases the resistance of the interference screws (200) to bending and torsional loads which may cause micromovements and possible reabsorption of bone around said screws; and
preserves the bone stock, preventing hyprepressure regions, which favors functional bone regeneration, in order to prevent periprosthetic fractures.

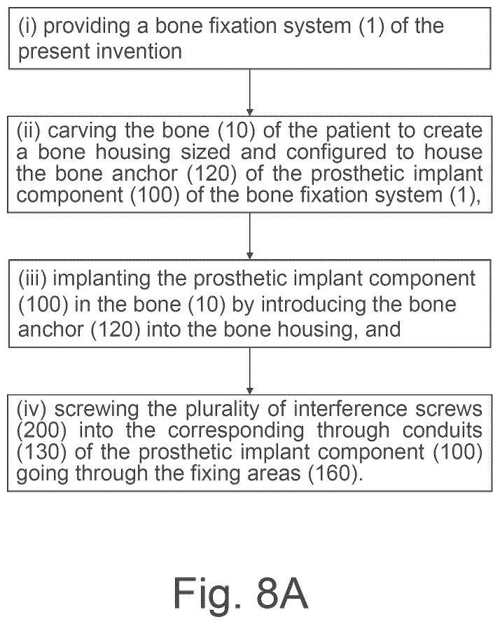

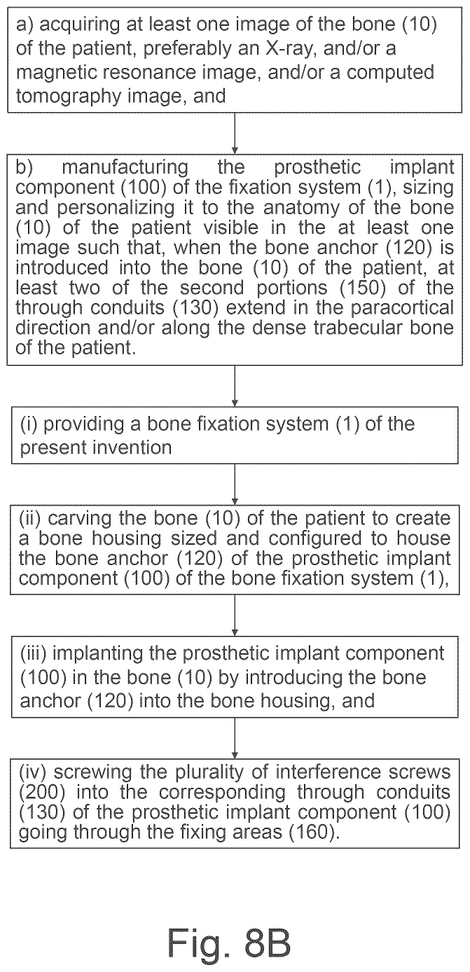

FIG. 8A shows a bone fixation method for fixing a prosthetic implant component (100) to a bone (10) of a patient in knee or hip arthroplasties, wherein said method comprises the following steps:
(i) providing a bone fixation system (1) such as that shown in FIGS. 2A-2E, 3A-3E, 5A-5D, and 6A-6D,
(ii) carving the bone (10) of the patient to create a bone housing sized and configured to house the bone anchor (120) of the prosthetic implant component (100) of the bone fixation system (1),
(iii) implanting the prosthetic implant component (100) in the bone (10) by introducing the bone anchor (120) into the bone housing, and
(iv) screwing the plurality of interference screws (200) into the corresponding through conduits (130) of the prosthetic implant component (100) going through the fixing areas (160).

A bone fixation system (1) such as the one described for the preceding figures is provided in step (i). The bone fixation system (1) provided in step (i) has the geometry and size corresponding to the geometry and size of the part of the bone to be replaced. In one embodiment, the prosthetic implant component (100) is standard or manufactured in a personalized manner.

In the second step, step ii), bone resection cuts, and/or drilling, and/or internal emptying, required for implanting the prosthetic implant component (100) in the bone (10), are performed on the bone (10) of the patient.

Then, in step iii), the at least one bone anchor (120) is housed, and/or impacted, and/or inserted in the inner volume created in the bone (10) of the patient in the preceding step ii).

In one embodiment, in order to increase the expansive force generated by screwing in the interference screws (200), the emptying of step ii) is performed such that, once the at least one bone anchor (120) is housed, and/or impacted, and/or inserted in the inner volume created in the bone (10) of the patient, the fixing areas (160) generated between the bone (10) and each of the open longitudinal portions (150) of the second portions (150) of the through conduits (130) comprise a diameter having a thickness that is less than the thickness of the diameter of the corresponding interference screw (200).

Lastly, in step iv), the interference screws (200) are screwed into the corresponding through conduits (130) by means of a screwdriver element (1000), going through the fixing areas (160) defined between the second portions (150) of each of the through conduits (130) and the cortical bone (10).

In one embodiment, for greater precision in the placement and fixation of the prosthetic implant component (100), the screwing in of the interference screws (200) is carried out alternately and progressively and/or using a dynamometric screwdriver element (1000).

### Embodiment in Knee Arthroplasty

In an example of knee arthroplasty, a bone fixation system (1) wherein the prosthetic implant component is a tibial component (100T) is provided in step (i).

In step (ii), dissection and dislocation of the joint is performed, exposing the tibia (10) to enable making bone resection cuts thereon which are necessary for arthroplasty. A bone housing suitable for housing the bone anchor (120T) is then created in the bone (10).

In a particular example, to create the bone housing, three bone fossae are created using a drilling template:
an anterocentral bone fossa 40 mm deep, using to that end a spiral drill 10 mm in diameter (a diameter that is 2 mm smaller than the diameter of the anterocentral interference screw);
a posteromedial bone fossa 30 mm deep, using to that end a spiral drill 7.5 mm in diameter (a diameter which is 1.5 mm smaller than the diameter of the posteromedial interference screw), and
a posterolateral bone fossa 30 mm deep, using to that end a spiral drill 7.5 mm in diameter (a diameter which is 1.5 mm smaller than the diameter of the posterolateral interference screw).

Advantageously, the use of spiral drills having a diameter smaller than the diameter of the corresponding interference screw (200) provides a greater screwing torque, such that the interferential and expansive force generated by screwing in the interference screws (200) increases, and therefore a greater primary stability of the implant is achieved.

Next, the three bone fossae are attached to one another by means of a reamer having a geometry similar to the geometry of the bone anchor (120T), thereby forming a bone housing suitable for inserting said bone anchor (120T).

In particular embodiments, these first two steps are carried out by using 3D imaging means, and/or virtual reality, and/or navigation systems, and/or robot-assisted surgery.

In step iii), the at least one bone anchor (120T) is impacted, and/or inserted, and/or fitted, and/or accommodated in the bone housing and/or bone holder created in the preceding step.

Advantageously, the fixation system (1) of the invention allows an initial fitting of the tibial prosthetic implant component (100T) by means of light tapping, and then fitting by means of screwing. The risk of periprosthetic fracture associated with hammering, which is necessary to achieve the primary fixation of uncemented implant components of the state of the art, is thus prevented.

Next, in step iv), each of the three interference screws (200) is screwed into its corresponding through conduits (130) by means of a screwdriver element (1000), going through the fixing areas (160).

In this particular example, each of the second portions (150) of the three through conduits (130) comprises a longitudinal truncated female thread portion (151), so each of the interference screws (200) goes through a fixing area (160) defined between the corresponding longitudinal truncated female thread portion (151) and the dense subcortical trabecular bone, without invading the cortex.

In preferred embodiments of the invention, the screwing in of the interference screws (200) is performed using a dynamometric screwdriver (1000), partially and successively changing from one interference screw (200) to another. A precise fitting of the prosthetic implant component in the bone (10) by means of screwing is thus achieved.

FIG. 8B shows the bone fixation method of FIG. 8A, but further comprising the following steps prior to steps (i)-(iv):
a) acquiring at least one image of the bone (10) of the patient, preferably an X-ray, and/or a magnetic resonance image, and/or a computed tomography image, and
b) manufacturing the prosthetic implant component (100) of the fixation system (1), sizing and personalizing it to the anatomy of the bone (10) of the patient visible in the at least one image such that, when the bone anchor (120) is introduced into the bone (10) of the patient, at least two of the second portions (150) of the through conduits (130) extend in the paracortical direction and/or along the dense trabecular bone of the patient.

In one embodiment, the manufacture of the prosthetic implant component (100) comprises the following three steps: in a first step, a prosthetic implant component (100) comprising two or more closed through conduits (130) is manufactured; in a second step, an inner thread is created in the two or more closed through conduits (130) with a female thread, and in a third step a distal portion of the two or more closed through conduits (130) is cut longitudinally. In this way, the generated through conduits (130) comprise first portions (140) formed as closed conduits with a female thread and second portions (150) formed as open conduits with a truncated female thread, with both thread types sharing one and the same helical trajectory.

1. A bone fixation system for fixing a prosthetic implant component to a bone of a patient in knee or hip arthroplasties, the bone fixation system comprising:
(a) a prosthetic implant component comprising:
(i) a prosthetic body comprising a first face configured to be coupled at least partially to a prosthetic insert and a second face configured to at least partially contact with the bone;
(ii) a bone anchor configured to be implanted in the bone, the bone anchor comprising (1) a proximal end attached to the second face of the prosthetic body, (2) a distal end, and (3) a plurality of through conduits each of which is oriented according to a longitudinal axis, each of the plurality of through conduits comprising:
(aa) a first portion formed as a closed conduit in the prosthetic body going through said prosthetic body from the first face to the second face; and
(bb) a second portion which is formed as an open conduit in the bone anchor defining a fixing area;
wherein:
(cc) the first portion and/or the second portion comprise a first threading region, and
(dd) the longitudinal axes of at least two of the plurality of through conduits form an angle greater than zero degrees and less than ninety degrees, the angle being closed in a proximal-distal direction; and
(b) a plurality of interference screws each of which:
(i) comprises a second threading region, complementary to the first threading region of a corresponding through conduit; and
(ii) is configured to be screwed into the corresponding through conduit;
wherein the bone anchor is configured to be implanted in a bone housing carved in the bone, and the prosthetic implant component is configured to be fixed to the bone with the interference screws screwed into the through conduits going through the fixing areas.

2. The bone fixation system of claim 1, wherein the second portion of each through conduit of the plurality of through conduits is formed as an open conduit having a cross-section that is in a shape of a circumference arch with an angle Φ equal to or greater than 180°.

3. The bone fixation system of claim 1, wherein the angle formed by the longitudinal axes of at least two through conduits of the plurality of through conduits is in a range from 5 degrees to 65 degrees.

4. The bone fixation system of claim 1, wherein:
the first portion of each through conduit comprises a proximal part and a distal part, wherein:
the distal part comprises the first threading region; and
the proximal part has a frustoconical section; and
each interference screw comprises a frustoconical head complementary to the proximal part of the first portion.

5. The bone fixation system of claim 1, wherein the secondary threading region of the plurality of interference screws is blunt, squared, rounded, or trapezoidal.

6. The bone fixation system of claim 1, further comprising:
a screwdriver element configured to screw the interference screws into the prosthetic implant component, and/or
a bone preparation element configured to carve the bone and create the bone housing sized and configured to house the bone anchor.

7. The bone fixation system of claim 1, wherein the plurality of interference screws comprise a metric of between 6 mm and 20 mm, and a length of between 15 mm and 100 mm.

8. The bone fixation system of claim 1, wherein:
the prosthetic implant component is a tibial prosthetic implant component;
the first face of the prosthetic body is configured to be coupled at least partially to a tibiofemoral insert and the second face is configured to contact a tibial bone resection plane;
the bone anchor comprises an anterocentral tibial piece, a posteromedial tibial piece, and a posterolateral tibial piece; and
the plurality of through conduits comprises an anterocentral through conduit, a posteromedial through conduit, and a posterolateral through conduit,
wherein the longitudinal axes of the posterolateral through conduit and the posteromedial through conduit form an angle between 30 and 65 degrees, and
the longitudinal axis of the anterocentral through conduit forms, with each of the longitudinal axes of the posterolateral through conduit and the posteromedial through conduit an angle between 15 and 45 degrees, and
wherein the plurality of interference screws is three interference screws.

9. The bone fixation system of claim 8, wherein the bone anchor is formed as a pyramid frustum or a cone frustum.

10. The bone fixation system of claim 1, wherein:
the prosthetic implant component is a knee femoral prosthetic implant component;
the first face of the prosthetic body is configured to be coupled at least partially to a tibiofemoral insert and the second face is configured to contact a femoral condyle bone resection plane; and
the plurality of through conduits comprise a medial through conduit and a lateral through conduit, wherein the longitudinal axes of the medial through conduit and of the lateral through conduit form an angle between 15 and 45 degrees; and
the plurality of interference screws is two interference screws.

11. The bone fixation system of claim 10, wherein the bone anchor comprises a piece formed as a pyramid frustum and having a base, and the piece, with the exception of the base, comprises four concave faces.

12. The bone fixation system of claim 1, wherein:
the prosthetic implant component is a hip femoral prosthetic implant component;
the second face of the prosthetic body is configured to contact a femoral epiphysis bone resection plane;
the first face of the prosthetic body is configured to be coupled at least partially to a femoroacetabular insert; and
the plurality of through conduits comprises a lateral through conduit and a posterior through conduit, wherein the longitudinal axes of the lateral through conduit and of the posterior through conduit form an angle between 5 and 45 degrees; and
the plurality of interference screws is two interference screws.

13. The bone fixation system of claim 1, wherein:
the prosthetic implant component is an acetabular prosthetic implant component;
the prosthetic body is an acetabular prosthetic body formed as an acetabular cup comprising a circumference flange comprised in plane π;
the second face of the acetabular prosthetic body is configured to contact an acetabular bone;
the first face of the acetabular prosthetic body is configured to be coupled at least partially to a femoroacetabular insert; and
the plurality of through conduits comprise an anterior through conduit, a medial-superior through conduit, a medial-inferior through conduit, and a posterior through conduit,
wherein the longitudinal axes of the medial-inferior through conduit and the medial-superior through conduit form an angle between 15 and 60 degrees, and
the longitudinal axis of the medial-superior through conduit forms an angle between 30 and 90 degrees with respect to plane π, and
wherein the plurality of interference screws is four interference screws.

14. The bone fixation system of claim 13, wherein the bone anchor comprises a piece formed as a pyramid frustum inclined with respect to the plane π, and the piece comprises eight concave faces in a medial cross-section.

15. The bone fixation system of claim 1, wherein the first portion formed as the closed conduit in the prosthetic body goes through said prosthetic body from the first face to the second face and partially through the bone anchor.

16. A bone fixation assembly for fixing the prosthetic implant components to the tibia and to the femur of a patient in knee arthroplasties, wherein the assembly comprises:
a first bone fixation system according to claim 1, wherein the prosthetic implant component is a tibial prosthetic implant component;
a second bone fixation system also according to claim 1, wherein the prosthetic implant component is a knee femoral prosthetic implant component; and
a tibiofemoral insert.

17. A bone fixation assembly for fixing the prosthetic implant components to the femur and to the pelvis of a patient in hip arthroplasties, wherein the assembly comprises:
a first bone fixation system according to claim 1, wherein the prosthetic implant component is a hip femoral prosthetic implant component;
a second bone fixation system also according to claim 1, wherein the prosthetic implant component is an acetabular prosthetic implant component; and
a femoroacetabular insert.

18. A bone fixation method for fixing a prosthetic implant component to a bone of a patient in knee or hip arthroplasties, wherein said method comprises the following steps:
(i) providing a bone fixation system according to claim 1;
(ii) carving the bone of the patient to create a bone housing sized and configured to house the bone anchor of the prosthetic implant component of the bone fixation system;
(iii) implanting the prosthetic implant component in the bone by introducing the bone anchor into the bone housing; and
(iv) screwing the plurality of interference screws into the corresponding through conduits of the prosthetic implant component going through the fixing areas.

19. The bone fixation method of claim 18, wherein further comprising, prior to performing steps (i)-(iv):
a) acquiring at least one image of the bone of the patient; and
b) manufacturing the prosthetic implant component of the fixation system, sizing and personalizing it to an anatomy of the bone of the patient visible in the at least one image such that, when the bone anchor is introduced into the bone of the patient, at least two of the second portions of the through conduits extend in a paracortical direction and/or along a dense trabecular bone of the patient.
